(19) United States
(12) Patent Application Publication
(10) Pub. No.: US 2025/0084396 A1
(43) Pub. Date: Mar. 13, 2025

(54) RAPIDLY-SEDIMENTING MAGNETIC PARTICLES AND APPLICATIONS THEREOF

(71) Applicant: Beckman Coulter, Inc., Brea, CA (US)

(72) Inventors: Thomas Marshall, Indianapolis, IN (US); Evan Farthing, Indianapolis, IN (US); Jeffrey J. Corpstein, Noblesville, IN (US); Thomas G. Keen, Indianapolis, IN (US); Abraham Olson, Indianapolis, IN (US); Asmita Patel, Carmel, IN (US); Amy Yoder, Brea, CA (US); Sahana Mallya, Union City, CA (US); Joseph Dominic Olechno, Hayward, CA (US)

(21) Appl. No.: 18/547,925

(22) PCT Filed: Feb. 24, 2022
(86) PCT No.: PCT/US2022/070828

§ 371 Date: Aug. 25, 2023

Related U.S. Application Data
(60) Provisional application No. 63/153,717, filed on Feb. 25, 2021.

(51) Int. Cl. C12N 15/10

(52) U.S. Cl. CPC C12N 15/1013

(57) ABSTRACT

According to various aspects of the instant disclosure, a rapidly-sedimenting magnetic particle can include a core or inner layer. The core or inner layer can include a ferrimagnetic material and have at least one of a maximum field strength ranging from about 20 emu/g to about 250 emu/g, and a remanence ranging from about 0 cmu/g to about 20 emu/g. The rapidly-sedimenting magnetic particle can further include a coating layer overlaying at least a portion of the core or inner layer. The particle can further include an outer coating layer overlaying at least a portion of the core coating.

### CLAIM OF PRIORITY

This patent application claims the benefit of priority to U.S. Application Ser. No. 63/153,717, filed Feb. 25, 2021, which is incorporated by reference herein in its entirety.

### BACKGROUND

Magnetic particles (e.g., paramagnetic and superparamagnetic particles) are used for sample analysis and preparation in a variety of contexts, including chemical and biological assays and diagnostics. Such paramagnetic and superparamagnetic particles have also been used in microfluidic systems. Magnetic particle technology is a robust technology that provides for high performance (e.g., device sensitivity and accuracy) and also provides for easy automation of assay protocols. In some applications, the surface of magnetic particles can be coated with a suitable ligand or receptor (e.g., antibodies, lectins, oligonucleotides, or other affinity groups), which can selectively bind a target substance or a group of analytes in a mixture. In some applications, the magnetic particles are used for mass transfer of components from one substrate to another substrate. One element in magnetic particle separation and handling technology is efficient mixing to enhance the reaction rate between the target substances and the particle surfaces, the mass transfer from one substrate to another, or the transfer of an analyte from one medium to another.

### SUMMARY OF THE INVENTION

According to various aspects of the instant disclosure, a rapidly-sedimenting magnetic particle can include a core. The core can include a ferrimagnetic material and have at least one of a maximum field strength ranging from about 20 emu/g to about 250 emu/g, and a remanence ranging from about 0 emu/g to about 20 emu/g.

According to various aspects of the instant disclosure, a rapidly-sedimenting magnetic particle includes a core comprising a ferrimagnetic material. A size of the rapidly-sedimenting magnetic particle is in a range of from about 75 nm to about 200 μm. A remanence of the rapidly-sedimenting magnetic particle is in a range of from about 0 emu/g to about 20 emu/g. A density of the rapidly-sedimenting magnetic particle is in a range of from about 1.1 g/cm³ to about 5.2 g/cm³.

According to various aspects of the instant disclosure, a rapidly-sedimenting magnetic particle includes a core including a ferrimagnetic material. A sedimentation velocity of the rapidly-sedimenting magnetic particle is in a range of from about 0.3 mm/s to about 50 mm/s, about 0.5 mm/s to about 20 mm/s, less than, equal to, or greater than about 0.3 mm/s, 5, 10, 15, 20, 25, 30, 35, 40, 45, or 50 mm/s. when subjected to a magnetic field having a strength in a range of from about 750 Oersted to about 14,000 Oersted. A gravitational sedimentation velocity of the rapidly-sedimenting magnetic particle can be in a range of from about 3 mm/s to about 5 mm/s about 3.5 mm/s to about 4/5 mm/s, less than, equal to, or greater than about 3 mm/s, 3.5, 4, 4.5, or about 5 mm/s. A remanence of the rapidly-sedimenting magnetic particle is in a range of from about 0 emu/g to about 20 emu/g.

According to various aspects of the instant disclosure, a density of the rapidly-sedimenting magnetic particle is in a range of from about 1.1 g/cm³ to about 5.2 g/cm³.

According to various aspects of the instant disclosure, a rapidly-sedimenting magnetic particle can include a core having ferrimagnetic material and having a maximum field strength ranging from about 20 emu/g to about 250 emu/g, a remanence ranging from about 0 emu/g to about 20 emu/g, or both.

According to various aspects of the instant disclosure, a kit includes a rapidly-sedimenting magnetic particle. The rapidly-sedimenting magnetic particle can include a core. The core can include a ferrimagnetic material and have at least one of a maximum field strength ranging from about 20 emu/g to about 250 emu/g, and a remanence ranging from about 0 emu/g to about 20 emu/g. The rapidly-sedimenting magnetic particle can further include a core coating layer overlaying at least a portion of the core. The particle can further include an outer coating layer overlaying at least a portion of the core coating.

According to various aspects of the instant disclosure, a method of processing a sample includes contacting the sample with a rapidly-sedimenting magnetic particle. The rapidly-sedimenting magnetic particle can include a core. The core can include a ferrimagnetic material and have at least one of a maximum field strength ranging from about 20 emu/g to about 250 emu/g, and a remanence ranging from about 0 emu/g to about 20 emu/g. The rapidly-sedimenting magnetic particle can further include a core coating layer overlaying at least a portion of the core. The particle can further include an outer coating layer overlaying at least a portion of the core coating.

According to various aspects of the instant disclosure, a method of processing a sample includes providing a rapidly-sedimenting magnetic particle. The rapidly-sedimenting magnetic particle can include a core. The core can include a ferrimagnetic material and have at least one of a maximum field strength ranging from about 20 emu/g to about 250 emu/g, and a remanence ranging from about 0 emu/g to about 20 emu/g. The rapidly-sedimenting magnetic particle can further include a core coating layer overlaying at least a portion of the core. The particle can further include an outer coating layer overlaying at least a portion of the core coating. The method further includes contacting a solution containing an analyte of interest with the rapidly-sedimenting magnetic particle. The method further includes subjecting the rapidly-sedimenting magnetic particle to a magnetic field, thereby allowing the rapidly-sedimenting magnetic particle to be separated from the solution. The method further includes collecting the rapidly-sedimenting magnetic particle at a collection site after the rapidly-sedimenting magnetic particle is exposed to a magnetic field. The method further includes isolating the rapidly-sedimenting magnetic particles. The method further includes eluting the analyte of interest from the rapidly-sedimenting magnetic particle. The method further includes performing a polymerase chain reaction procedure on the analyte of interest.

### BRIEF DESCRIPTION OF THE FIGURES

The drawings illustrate generally, by way of example, but not by way of limitation, various embodiments of the present invention.

FIG. 1 is a graph that shows that magnetic beads can concentrate virus from a larger volume of material.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

A need exists to improve the overall speed and efficiency of separation using rapidly-sedimenting magnetic particles. A need also exists for rapidly-sedimenting magnetic particles that have a high response to external magnetic fields as well as a low remanence.

The present teachings generally relate to sample processing methods and systems for mixing, separating, filtering, or otherwise processing a sample (e.g., a fluid sample) by utilizing rapidly-sedimenting magnetic particles (e.g., ferrimagnetic particles) that are caused to move under the influence of a magnetic field. Although rapidly-sedimenting magnetic particles such as ferrimagnetic particles are described in conjunction with numerous embodiments, aspects, and examples in accordance with the instant disclosure it is also contemplated that rapidly-sedimenting magnetic particles such as ferromagnetic particles, paramagnetic particles, and superparamagnetic particles, or mixtures of various classes of rapidly-sedimenting magnetic particles can also be used. Therefore, any specific recitation of a ferrimagnetic particle can be equally applied to a ferromagnetic particle, paramagnetic particle, superparamagnetic particle, or mixtures thereof.

### Rapidly-Sedimenting Magnetic Particles

Magnetic particles are commonly used in sample isolation or processing steps because they allow analytes (e.g., molecules) of interest, attached or bound to the magnetic particle, to be easily separated from other (soluble) molecules or components in a sample solution by pulling-down the magnetic particles using magnetic forces to form a pulled-down solid phase separate from a supernatant liquid phase. Pouring-off or aspirating-off the supernatant liquid effectively separates the analytes of interest associated with the particles from the other molecules, liquid, or components removed together with the supernatant.

In such processes or applications using magnetic particles for the separation of target analytes of interest in a sample solution, the amount of time it takes to pull-down the magnetic particles may be a rate-limiting step. The rapidly-sedimenting magnetic particles of the present disclosure have physical and magnetic properties that may allow them to be pulled-down faster, (e.g., in a shorter time period), compared to typical magnetic particles used in the prior art. This faster pull-down may therefore lead directly to a faster assay, purification, or processing step with a shorter turn-around time and/or higher throughput compared to that using other magnetic particles. Physical and magnetic properties leading to a faster pull-down may include the size, density, sedimentation rate, magnetic field strength, and/or remanence of the magnetic particle.

Larger and/or more dense particles tend to naturally settle or sediment faster than smaller and/or less dense particles. This higher settling or sedimentation rate may contribute to a faster pull-down rate of the particles when subject to an external magnetic field. Magnetic particles having a higher magnetic field strength tend to be more responsive to an external magnetic field, thus being pulled down faster than other particles having a lower magnetic field. Magnetic particles having a relatively low remanence tend to resist clumping or self-associating in the absence of an external magnetic field, whereas particles having a higher remanence (i.e., higher residual magnetism), may tend to clump together when the external magnetic field is removed due to the self-magnetic-attraction of the magnetic particles. This clumping may cause the particles to clog or block a pipettor or dispenser used to handle, transfer, aspirate, or dispense the particles, making these particles unsuitable for certain common processes using liquid-handler devices. This clumping may also interfere with the binding of the particles to target molecules or analytes of interest in solution by effectively reducing the surface area of the particles.

Several general types of magnetic particles have been used in the prior art. In some cases, paramagnetic or superparamagnetic particles have been used. These particles tend to have a lower magnetic field strength compared to ferromagnetic (i.e., permanent-magnet) particles, but have the advantage of a relatively low remanence. These particles also tend to be chosen or designed to have a minimal sedimentation rate, so that the particles remained evenly distributed or suspended in solution during binding and incubation reactions in order to maximize interaction with target molecules during these steps

In other cases, ferromagnetic particles having a relatively high magnetic field strength have been used, including ferromagnetic particles having a density of approximately 1.8 g/cm³ (U.S. Pat. No. 9,790,539). Such ferromagnetic particles, however, tend to have a correspondingly higher remanence, which may make them then unsuitable for processes using common liquid-handler devices.

The sedimentation rate of a particle may be characterized as the time it takes for a vast majority of the particles to settle to the bottom of a vessel or container, starting with a uniform suspension of the particles in the vessel or container. In some cases, the sedimentation rate may be characterized as the time it takes all visually discernable or identifiable particles to reach the bottom of the vessel or container. Characterized as such, the sedimentation rate may be dependent not only on the size and density of the particles, but also on the density or viscosity of the solution and of the height of the liquid level in the vessel or container.

### Gravity-Assisted Magnetic Capture

Typical magnetic particles used for the isolation or other processing of target analytes in solution are pulled-down or captured using essentially magnetic forces only. In fact, such particles for these processes are typically chosen, designed, or selected to have a minimal sedimentation rate so that the particles remain evenly distributed in solution, providing the highest possible surface area for the most efficient binding or interaction with target molecules in solution. However, the magnetic force of the magnet(s) used to pull-down the particles in a container is inversely proportional to the square of the distance between the magnet and the particle. This limitation therefore limits the size of the container and/or the volume of solution that can be used for magnetic capture using typical magnetic beads.

The rapidly-sedimenting magnetic particles of the present disclosure have a natural tendency to sink or sediment to the bottom of a vessel or container, thus assisting in the magnetic capture of the particles using a magnet disposed towards the bottom of the vessel or container. This allows effective magnetic capture of the magnetic particles from a large volume, where gravitational forces, acting throughout the vessel or container, will bring the particles into range of the magnetic forces near the bottom of the vessel or container. In some examples, the bottom of the vessel or container may be sloped so that the magnetic particles naturally settle onto one or more small-area capture zones of the vessel or container, where the capture/pull-down magnet may be located. The magnetic particles (and any target molecule or reagent attached thereto) may be separated from the supernatant (e.g., the remaining sample solution) by pouring-off or otherwise removing the supernatant, or by removing the magnetic particles from the capture zone, such as through an outlet coupled to the capture zone. A valve may be used to isolate the capture zone from the rest of the sample solution in the vessel or container. In some examples, the capture zone may be in a recess or chamber embedded in a sidewall of the vessel or container. Gravity-assisted magnetic capture may be performed while the sample vessel or container is tilted such that the capture zone is at the lowest point of the vessel or container. The magnetic particles may then be isolated or removed from the remaining sample solution (i.e., supernatant) by tilting the vessel or container such that the magnetic particles in the capture zone remain in the recess or chamber in the sidewall. A valve and/or outlet may be used to facilitate removal of the captured magnetic particles from the vessel or container.

In some examples, an analyte-specific sensor may be coupled to the capture zone to detect and/or quantitate an analyte pulled-down onto the capture zone via the magnetic particle.

Because gravity is not affected by the size of a container or the volume of liquid in the container, gravity-assisted magnetic capture may be used to effectively detect, isolate, or quantitate a target molecule present in trace amounts in a large volume, (including hundreds of liters, or more), using rapidly-sedimenting magnetic particles of the present disclosure.

### Maintaining Magnetic Particles in Suspension

The rapidly-sedimenting magnetic particles of the present disclosure tend to naturally settle to the bottom of a sample vessel or container. However, it is often desirable to maintain the magnetic particles evenly distributed in a sample solution during a binding, enzymatic, hybridization, or other reaction or incubation. In one example, the rapidly-sedimenting magnetic particles of the present disclosure may be maintained in suspension during such reaction or incubation by agitating the sample solution containing the magnetic particles. This agitation may be accomplished, for example, by stirring the solution, or by rocking or shaking the vessel containing the solution, during the binding or other reaction.

In another example, the rapidly-sedimenting magnetic particles of the present disclosure may be maintained in suspension by periodically pipetting the solution up and down using a pipettor.

In another example, the rapidly-sedimenting magnetic particles of the present disclosure can be maintained in suspension by applying a changing or oscillating external magnetic field to induce a mixing movement or motion of the magnetic particles. In some examples, the magnetic particles have physical and magnetic properties making them suitable for mixing using an electromagnetic device, thus automatically maintaining the particles in suspension using a device having no moving parts.

### Use of Magnetic Forces to Mix, Move, Resuspend, Heat, or Transfer a Sample Solution or a Target Molecule or Object Suspended Therein in Solution

The movement of rapidly-sedimenting magnetic particles of the present disclosure can be induced by the application of a changing or oscillating external magnetic field. Such a changing or oscillating magnetic field may be applied, for example, using moving permanent magnets. In other examples, the changing or oscillating magnetic field may be applied using an arrangement of electromagnets surrounding a vessel containing a sample solution including the magnetic particles, such as described in WO 2020/018919. In this way, the movement of the particles can be induced without the need for magnets to physically rotate about the vessel container, thereby providing a simpler, more efficient, and more robust mixing device. In some examples, the induced movement of the magnetic particles may be used to maintain the magnetic particles in suspension to facilitate interaction or binding of the magnetic particles to the target molecule.

In some examples, the induced movement of the magnetic particles in the sample solution may also be used to mix the sample solution itself, thereby dispersing the target molecules dissolved or suspended therein. This mixing and dispersing may further facilitate the interaction or binding of the target molecules with the magnetic particles or with any other molecule, reagent, or component in the sample solution.

In some examples, induced movement of the magnetic particles may cause stirring or mixing of the liquid solution containing the target sample molecules. In some examples, the target molecules may be reversibly or irreversibly attached or bound to the magnetic particles, such that the induced movement of the magnetic particles leads to the corresponding direct movement of the target molecules, for mixing, resuspension, transport, or the like.

In some examples, the induced movement of the magnetic particles may create a flow stream in the sample solution, causing sample molecules or objects of interest to be transported or moved together with the magnetic particles in the stream. In some examples, target molecules or objects may become bound or otherwise attached to the magnetic particles via a target-specific ligand attached to the particle, or by any other suitable means. In these examples, movement of the target molecules or objects is directly tied to the movement of the magnetic particles in response to the changing magnetic field.

The induced movement of the target molecules or objects may be used to move the target molecules from a first location to a second location, where the physical, chemical, or other conditions may differ between the two locations. For example, the target molecules or objects may be subject to a first temperature at the first location, and a second temperature at the second location, in order to process the target molecules or objects. In another example, the target molecules or objects may be exposed to a first reagent at the first location, and to a second reagent, (or more generally to a lack of the first reagent) at the second location, in order to process the target molecules or objects. In another example, the target molecules or objects may be moved from a first location where the target molecules are incubated in a binding or other processing or assay reaction, to a second location where bound or processed target molecules or objects are detected, isolated, quantified, and/or analyzed.

The induced movement of the target molecules or objects may be directional, based on the directionality of the applied magnetic fields. In this way, the movement of the target molecules may be guided in a distinct three-dimensional path, allowing transport of the target molecules or objects from one location, chamber, substrate, channel, or the like to another, including directing the proper turn at any junction or branchpoint in the path.

The induced movement of the target molecules may be used simultaneously and/or in combination with any other use of the rapidly-sedimenting magnetic particles described in this disclosure. For example, magnetic particles having a target-specific ligand may be used to bind and isolate a target molecule or object of interest in a sample solution. During the binding reaction, the magnetic particles may also be used to mix the sample solution for a more efficient binding reaction, and/or to disperse the magnetic particles to keep them in suspension during the binding reaction.

In some examples, the changing magnetic fields applied to the sample solution may be used to heat the sample or to otherwise control the temperature of the sample. The heat may be generated, for example, from the kinetic energy of the moving magnetic particles, or may be generated, for example, as resistive heat resulting from the electrical current used to activate electromagnets that produce the changing magnetic fields. In other examples, the magnetic mixing device used to generate the external magnetic fields that induce movement and/or mixing of the magnetic particles may comprise a separate heating (and/or cooling) element or device to control the temperature of the sample solution.

In some examples, the sample solution may be actively mixed by the magnetic particles while heating or otherwise adjusting or maintaining the temperature of the sample solution. The mixing may provide more rapid heating and more even distribution of the heat, instead of relying on relatively slow convection mixing of cold and hot areas. The combination of mixing while heating or maintaining an elevated temperature may be used, for example, to maintain a more constant temperature when incubating a hybridization reaction for the capture of desired NGS target fragments. In another example, the mixing may provide a more mild or consistent heating during a ligation reaction.

The mixing by the movement of rapidly-sedimenting magnetic particles of the present disclosure induced by a changing external magnetic field may have the advantage of being more gentle and less disruptive than other common methods of mixing, such as by vortexing. For example, samples mixed using the rapidly-sedimenting magnetic particles of the present disclosure in a changing magnetic field may generate fewer bubbles, causing less disruption of sample molecules in the solution, while generating less splashing, which may lead to sample loss and contamination, compared to samples mixed using a vortex mixer. In addition, mixing using the rapidly-sedimenting magnetic particles of the present disclosure may be more efficient compared to other physical methods of mixing, such as rocking or shaking the sample vessel, or my stirring the solution in the sample vessel.

The efficiency of mixing the sample solution using rapidly-sedimenting magnetic particles of the present disclosure in a changing magnetic field may be enhanced based on the size of the magnetic particles. Larger particles, for example, may provide more efficient mixing of the sample solution. In some examples, the rapidly-sedimenting magnetic particles of the present disclosure may comprise two populations of magnetic particles. One population may have a smaller average diameter and may include a ligand for specific binding or interaction with the target molecule, while the other population may have a larger average diameter but no ligand, to provide more efficient mixing of the sample solution resulting in more efficient binding or interaction of the smaller population of magnetic particles with the target molecules.

In some examples, the rapidly-sedimenting magnetic particles of the present disclosure exposed to a changing magnetic field can be used to mix and/or resuspend a lyophilized sample into solution. The magnetic particles may include a surface ligand to bind or interact with a target molecule in the resuspended solution for processing the resuspended sample. In this way, the rapidly-sedimenting magnetic particles of the present disclosure May provide a more efficient mixing/resuspension of the sample, as well as combining resuspension with a further sample prep or processing step.

Physical and magnetic properties of the rapidly-sedimenting magnetic particles of the present disclosure making them suitable for mixing in an electromagnetic device can include an average density of about 1.1 to about 5.2 g/cm³, an average diameter of about 75 nm to about 1 μm, a maximum field strength (alternatively known as a “saturated magnetic response”) of about 35 emu/g to about 100 emu/g, and a remanence of about 0 emu/g to about 20 emu/g.

### Microfluidic Devices

The controlled, induced movement of a sample solution or of a target molecule or object suspended or dissolved in the sample solution as described above may be particularly useful in a microfluidic cartridge or device, where rapidly-sedimenting magnetic particles may be used to move or otherwise transport fluids, or target molecules or objects in the fluids, from any site, position, chamber, channel, port, valve, intersection, junction, or any other location in the device to a different site, position, chamber, channel, port, valve, intersection, junction, or location in the device.

### Concentrating or Enriching a Target Molecule in Solution

In some applications, rapidly-sedimenting magnetic particles of the present disclosure can be used to more quickly or efficiently concentrate target molecules in a solution. For example, the magnetic particles can include a surface ligand that reversibly or irreversibly binds to the target molecule. The magnetic particles can then be used to bind to the target molecules in a dilute sample of the target molecules. Pulling-down the magnetic particles with magnetic forces, and then removing all or a portion of the resultant supernatant solution leads to a more concentrated sample of the target molecules. Elution of reversibly-bound target molecules from the magnetic particles into a small volume and separation of the particles from the eluted target molecules can lead to a more concentrated sample of free target molecules. In some examples, the magnetic particles include a surface ligand that irreversibly binds to the target molecule to produce the more concentrated solution of immobilized target molecules.

The target molecules concentrated or enriched by these methods can include any target molecule described herein, including any nucleic acid, protein, carbohydrate, lipid, cell, virus, organelle-component, cell component, tissue-component, virus, microbe, pathogen, pollutant, contaminant, poison, analyte, diagnostic marker, or the like.

The sample solutions from which the target molecules are concentrated or enriched can be any sample solution described herein, including any biological, clinical, environmental, forensic, agricultural, or water sample, or the like.

The rapidly-sedimenting magnetic particles of the present disclosure may be particularly useful for the concentration or enrichment of target molecules in dilute samples or large volumes using gravity-assisted magnetic capture, as described earlier.

Concentrating or enriching a target analyte molecule containing a detectable label may be used to detect or visualize the label, thereby detecting the target molecule. For example, in the case of a visual label, such as a colored dye or chromophore, the concentration of the dye or chromophore in the sample concentrated using the magnetic particles may exceed a threshold such that the dye or chromophore becomes visible to the naked eye, indicating a positive test result, (i.e., presence of the target analyte in the sample). Such a method may be used, for example, in a home pregnancy test. In other examples, the label may be a fluorophore or any other suitable label. In some examples, the labeled target molecules, bound to magnetic particles, may be pulled-down to a relatively small pellet, thereby providing a high local-concentration of the label to facilitate detection.

### Isolation, Detection, Quantitation, Processing, or Characterization of a Target Molecule in a Sample

Rapidly-sedimenting magnetic particles of the present disclosure may be used to more quickly and/or efficiently pull-down and thereby isolate/detect a bound target molecule from a sample solution. As described below, the target molecule may be a nucleic acid, a protein, a carbohydrate, a lipid or steroid, a metabolite, an organelle, a cell, a microbe, a viral particle, a pathogen, a drug or other small chemical, a reagent or other processing molecule, a pollutant, a contaminant, a chemical reactant or product, a product of manufacture, or the like.

The magnetic particle may have a ligand molecule attached to the outer surface of the particle, that specifically or non-specifically binds to the target molecule. The binding may be by covalent, ionic, hydrogen-bonding, polar, nonpolar, stearic, van der Waals forces, adsorption, or any other chemical or physical interaction. The magnetic particles may be added to the target-containing solution and incubated to allow the binding of target molecules to the magnetic particles. The incubation can be performed at any suitable temperature or pressure. The binding of the target molecules to the magnetic particles may be enhanced or accelerated by mixing the particles in the solution by applying a changing, alternating, or oscillating external magnetic field. The bound target molecules may then be separated from the rest of the solution by pulling-down the magnetic particles using a magnet, and removing the resulting supernatant solution. Alternatively, the bound target molecules may be separated and/or removed from the other solution components by inserting a magnet into the solution to capture the magnetic particles, and then removing the magnet, together with the magnetic particles and bound target molecules, from the solution. In some examples, the binding of target molecules to the magnetic particles is reversible, so that the target molecules may be recovered from the magnetic particles by resuspending the particles in an appropriate elution buffer. The free target molecules may then be separated from the particles using a magnet to pull-down or pellet the particles, or to remove the particles directly.

In some examples, target molecules of interest may be isolated from a sample solution using the rapidly-sedimenting magnetic particles of the present disclosure as a step in purifying or partially purifying the target molecules. In other examples, the target molecule is an undesired contaminant that is removed from a sample using the magnetic particles.

The rapidly-sedimenting magnetic particles of the present disclosure may be used to detect a target molecule in a sample by detecting the binding of the target molecule to the magnetic particle by any suitable means. In some examples, binding of target molecules to the magnetic particles, followed by concentrating the magnetic particles using magnetic forces, such as by pelleting or pulling-down the beads from solution, may lead to the visualization of a label associated with the target molecule, (that is undiscernible when dispersed in solution). In some examples, the target molecule may be detected by binding the target to the particle, removing the particle from the sample solution, and then detecting the target molecule bound to or subsequently released from the particle, by any suitable assay or detection means, including any suitable optical, chemical, electrical, electrochemical, physical, magnetic, biological, or other means. In some examples, the magnetic particles may be used to bind to the target molecule, such as by using a target-specific surface ligand, and then transported to a detection zone, such as by using magnetic forces, where any suitable target-specific assay or test may be performed. In some examples, the transportation of the target molecule to the detection zone may lead to a high local concentration of target molecule, facilitating detection and leading to a more sensitive assay for the target molecule.

The rapidly-sedimenting magnetic particles of the present disclosure may be used to quantitate the target molecules in a sample by quantifying the amount of target isolated or detected using the magnetic particles, using any suitable quantitation means. Similarly, the rapidly-sedimenting magnetic particles of the present disclosure may be used to otherwise characterize the target molecules that are bound, detected, or isolated using the particles, using any suitable means.

The rapidly-sedimenting magnetic particles of the present disclosure may be used to alter, modify, or otherwise process a target molecule of interest. In some examples, the magnetic particle may include, attached to its surface, a ligand that alters, modifies, or in any other way processes or interacts with the target molecule. In some examples, the ligand may be an enzyme, such as a nuclease, protease, kinase, ligase, phosphatase, ATPase, lipase, or any other enzyme. In one example, the enzyme may be proteinase k, such that incubation of a cellular sample with the proteinase k-coupled magnetic particles may lead to protease digestion of cellular components in the sample, resulting in cell lysis. In another example, the enzyme may be trypsin, such that incubation of a protein sample with the trypsin-coupled magnetic particles may lead to protease digestion of the protein sample, generating, for example, trypsin fragments for subsequent analysis by mass spectrometry. In some examples, the ligand may comprise two or more different enzymes. In one example, the ligand may comprise a cocktail of nucleases, such that incubation of the nuclease-coupled particles with a genomic DNA sample may lead to fragmentation of the genomic DNA to provide a starting material for NGS library preparation. In another example, the ligand may comprise a combination of proteinase k and a cocktail of nucleases, such that incubation of a cellular sample with the enzyme-conjugated particles may lead to both cell lysis and genomic DNA fragmentation in a single step. In yet another example, the ligand may comprise a combination of proteinase k, a cocktail of nucleases, and a carboxyl group, such that incubation of a cellular sample with the enzyme-conjugated, carboxylated particles may lead to cell lysis, genomic DNA fragmentation, and selective binding to the DNA fragments, all in a single step.

In some examples, the ligand may be a chemical catalyst for use in any suitable chemical reaction or process. In other examples, the ligand may be a cell-signaling molecule, such as a kinase, phosphatase, or hormone, such that incubation of the ligand-coupled particles to an in vivo, in situ, or in vitro cellular sample may lead to activation of a cellular or metabolic pathway. For example, the ligand may be a mitogen, such as epidermal growth factor, such that incubation of the sample with the ligand-conjugated beads may lead to activation of a mitogen-activated protein kinase pathway in the cellular sample.

In some examples, the ligand is any other chemical or biological reagent or agent that interacts with a target molecule of interest. In some examples the ligands can be specific to a particular analyte of interest and the magnetic particle including the ligand can be disposed in a sample to determine if the analyte of interest is present, in such a manner the magnetic particle can act as a probe or analytical device. In some other examples, the ligand may not bond to a specific analyte of interest, but instead may bond to classes of analytes such as an analyte having a carbonyl moiety, amine moiety, or the like. In that manner the magnetic particle can generally identify a class of analyte that can be present.

In all these examples, the ligand-coupled magnetic particles may be easily separated and/or isolated from the sample solution after the processing reaction using magnetic forces, so that the sample is ready for further processing without interference from the ligand molecule.

### Isolation, Detection, Quantitation, Processing, or Characterization of a Target Nucleic Acid in a Sample

Rapidly-sedimenting magnetic particles of the present disclosure may be used in a variety of applications involving the isolation, detection, quantitation, processing, or characterization of a target nucleic acid in a sample. Such applications may include polymerase chain reaction (“PCR”) amplification reactions, other thermal-cyclic or isothermal amplification reactions, ligation reactions, endonuclease or exonuclease digestion reactions, kinase reactions, phosphatase reactions, or any other enzymatic or nonenzymatic nucleic acid modification reaction. PCR amplification reactions may include real-time or end-point quantitative PCR, or digital PCR, including droplet-based digital PCR reactions. The magnetic particles may be used in the preparation or isolation of the target nucleic acid prior to the reaction, or in the isolation, processing, or characterization of the target nucleic acid after the reaction.

The target nucleic acid may be deoxyribonucleic acid (“DNA”) cDNA, ribonucleic acid (“RNA”), peptide nucleic acid (“PNA”), or any hybrid of these, either naturally-occurring, genetically engineered, or synthetic, including any enzymatically- or chemically-modified form thereof. The target nucleic acid may be single-stranded, double-stranded, or triple-stranded, and may include stem-loop or any other tertiary structure.

In some examples, the target nucleic acid may be present in low-abundance in a sample, such as cell-free DNA in a blood sample, including cell-free fetal DNA in a maternal blood sample.

The target nucleic acid molecule may be present in a whole cell, a biological fluid, a cell culture medium, a crude extract of a cell, a partially-purified fraction of a cellular extract, a purified fraction of a cell or cell extract, or the like.

The size of the target nucleic-acid molecule may be in the range of tens to hundreds of megabase pairs or more (for genomic DNA, for example), down to about a dozen or fewer nucleotides, (such as for an oligonucleotide).

Target DNA molecules may include genomic DNA; chromosomal DNA; chromatin; organellar DNA; circulating cell-free DNA, including fetal DNA and cancer- or tumor-related DNA; plasmid DNA; enzymatically-synthesized or amplified DNA; cDNA; DNA extracted or isolated from a cell, tissue, biological fluid, or organism; bacterial DNA, viral DNA; synthetic DNA, or the like, including any modified, tagged, or labelled form thereof. The target DNA molecule may be a segment or fragment of a larger DNA molecule, the target DNA molecule generated by cleavage, digestion, or fragmentation of the larger molecule, by physical, sonic, acoustic, enzymatic, or any other suitable means. Alternatively, the target DNA molecule may be generated by combining smaller DNA molecules by enzymatic, synthetic, or any other means.

Target RNA molecules may include mRNA; rRNA; tRNA; viral RNA;

microRNA; inhibitory or interfering RNA; in vitro transcribed or otherwise enzymatically-synthesized RNA, synthetic RNA; or the like, including any modified, tagged, or labelled form thereof.

The rapidly-sedimenting magnetic particles of the present disclosure may include a target-specific ligand attached to the surface of the particle. The target-specific ligand may selectively, or non-selectively/non-specifically bind to the target molecule. The binding may be reversible or irreversible. The binding may be by any suitable covalent, ionic, hydrogen-bonding, polar, nonpolar, stearic, van der waals, or other chemical or physical interaction. The target-specific ligand may be, for example, a nucleic acid having a sequence that is complementary to the target nucleic acid, whereby the magnetic particle will specifically bind to the target nucleic acid by nucleic-acid hybridization of complementary Watson & Crick base pairing. The bound target nucleic can then be separated from the sample solution using magnetic forces to pull and retain the magnetic particles to an internal surface of the sample vessel containing the sample solution. In other examples, the target nucleic is tagged with an affinity tag or label, and the rapidly-sedimenting magnetic particles of the present disclosure contain a surface ligand that specifically binds to the affinity label. The surface ligand can be, for example, avidin or streptavidin for the specific binding to biotin-tagged target nucleic acids. In other examples, the affinity-tag/ligand pair can be any pair of specific-binding molecules, such as antibody/antigen, polyhistidine/nickel, or the like. In other examples, the ligand is a carboxyl group for the selective binding to target nucleic acids in the presence of a crowding or precipitating reagent. In further examples, the ligand can include a carboxylic acid group, an amine group, an azide group, an alkane group, an alkene group, an alkyne group, a maleimide group, an acrylate group, an aldehyde group, a sulfhydryl group, an N-hydroxysuccinimide (NHS) activated group, an epoxy group, a hydroxyl groups, a sulfide, biotin, a bis-sulfone, a catechol, a cyclopentadiene, a norbornene, or a mixture thereof for the selective binding to target nucleic acids in the presence of a crowding or precipitating reagent.

Applications can include sequencing of the target nucleic acid, including dideoxy (Sanger) sequencing reactions or highly-multiplexed next-generation sequencing reactions (NGS). Rapidly-sedimenting magnetic particles of the present disclosure can be used, for example, in the isolation of template nucleic-acid molecules for these sequencing reactions, such as the isolation of high-molecular-weight DNA fragments for long-read sequencing. In other examples, the magnetic particles can be used for the isolation of sequencing fragments produced by the sequencing reaction.

Applications can include the assembly or construction of genomic elements for insertion into a microbe or cell, (i.e., synthetic biology). Rapidly-sedimenting magnetic particles of the present disclosure can be used, for example, in the isolation or clean-up of any intermediate or product nucleic acid produced as part of the synthetic biology process.

Applications can include gene-editing applications, including the use of CRISPR enzymes to create any particular genetic construct. The rapidly sedimenting magnetic particles can be used, for example, in the isolation, clean-up, processing, or characterization of any nucleic-acid intermediate or product in the CRISPR or any other gene-editing process.

Applications can include NGS library construction processes, where the rapidly-sedimenting magnetic particles of the present disclosure can be used, for example, in the clean-up, isolation, processing, or characterization of any intermediate- or product-nucleic-acid produced in the library-construction process, including any intermediate or product generated by reverse transcription, DNA fragmentation, end-repair, adapter-ligation, barcode-attachment, hybrid selection or capture, or amplification. Rapidly-sedimenting magnetic particles of the present disclosure can be used, for example, for faster and/or more efficient isolation or clean-up of any such intermediate or product, leading to an overall increase in the speed, throughput, and/or efficiency of the total NGS library construction process. In some examples, the isolated intermediate or product is quantified in order to normalize the different nucleic-acid samples or pools used in the NGS library construction.

Applications can include microarray analyses of target molecules in a sample. Rapidly-sedimenting magnetic particles of the present disclosure can be used, for example, for the faster and/or more efficient isolation, purification, or clean-up of the target molecules used for hybridization or binding to the microarray, thereby improving the speed, throughput, and/or sensitivity of the microarray analysis.

Applications can include an isolation, detection, and/or analysis of molecules that may be associated with or that bind to the target molecule. For example, rapidly-sedimenting magnetic particles of the present disclosure may be used for the isolation of various target molecules, such as high-molecular-weight chromatin or genomic DNA molecules, microRNA molecules, or extravesicular or microsomal particles, for the detection and/or analysis of lipid, protein, or nucleic acid components that co-isolate or purify with the target molecules.

Applications can include the isolation of nucleic acids using SPRI technology. In SPRI, a solution containing a crowding or precipitating reagent, such as a combination of polyethylene glycol (PEG) and salt, and magnetic particles having a carboxylated ligand attached to the surface (or other ligand as described herein), are added to the sample. Nucleic acids in the sample are selectively immobilized onto the magnetic particles in the presence of the crowding reagent. The immobilized nucleic acids are separated and isolated from the sample solution using a magnet to pull-down and retain the magnetic particles onto an inner surface of the sample container and removing the supernatant. The immobilized nucleic acids are then eluted off the magnetic particles by adding a low-salt solution, such as water. Before elution, the magnetic particles may be washed with a low-salt solution, such as ethanol. Because the size range of the nucleic acids that are selectively immobilized onto the magnetic beads is dependent on the concentration of crowding reagent in the sample solution, a desired size range of nucleic acid fragments can be isolated. For example, to isolate an intermediate size-range of fragments, a first PEG/salt concentration may be added to a sample solution to selectively bind fragments larger than the intermediate size-range, and removed from the solution using a magnet. Then, a second, higher PEG/salt concentration may be added to the solution to selectively bind the desired intermediate size range of fragments in the solution. These fragments may be separated from the smaller unbound fragments remaining in the solution by isolating the magnetic particles with a magnet. The bound intermediate size-range fragments may then be eluted off the breads with a low-salt buffer, such as water.

Other precipitating or crowding reagents that may be used with the SPRI method include ethanol, isopropanol, any other suitable alcohol, dextran, or a polycationic reagent, such as hexamine, ethylenediamine, polyamine, spermine, spermidine, or the cationic detergent cetyl trimethylammonium bromide (CTAB). These precipitating or crowding reagents may be used in combination with a salt, such as sodium chloride or sodium acetate, or any other suitable salt.

Rapidly-sedimenting magnetic particles of the present disclosure containing a surface carboxyl group (or other ligand as described herein) as ligand can be used for size selective isolation of nucleic acids from a sample using the SPRI method. Because the rapidly-sedimenting magnetic particles of the present disclosure may be pulled-down faster than traditional paramagnetic or superparamagnetic particles, the SPRI method using such rapidly-sedimenting magnetic particles of the present disclosure may be faster and/or more efficient, particularly for dilute nucleic acid samples. For example, carboxyl-coated, rapidly-sedimenting magnetic particles of the present disclosure can be used to isolate PCR products from a PCR reaction, using a PEG/salt concentration that will selectively immobilize the larger amplified nucleic-acid products while leaving the smaller nucleic-acid primers and free nucleotides (e.g., PCR reagents) in solution. In another example, the carboxyl-coated, rapidly-sedimenting magnetic particles of the present disclosure can be used to isolate a desired size range of fragmented nucleic acid fragments from smaller and larger molecules for NGS library construction, as described immediately above. More generally, rapidly-sedimenting magnetic particles of the present disclosure can be used for the faster and/or more efficient isolation or clean-up of target DNA or RNA molecules from cell or tissue lysates, from in vitro molecular biological reactions, and the like.

### Isolation, Detection, Quantitation, Processing, or Characterization of a Target Protein in a Sample

Rapidly-sedimenting magnetic particles of the present disclosure may be used for the isolation, detection, quantitation, processing, or characterization of a target protein in a sample. In some examples, the target protein may be an antibody. The antibody may be a native antibody or a fragment of a native antibody, or any enzymatically- or chemically-modified form of such an antibody or antibody fragment. The antibody may be a natural, synthetic, or genetically-engineered molecule. The antibody may be monoclonal or polyclonal. The magnetic particle may have, attached to its surface, a ligand that specifically or non-specifically binds to the target antibody. In some examples, the ligand is an antibody-specific binding molecule, such as Protein A, or a species-specific anti-immunoglobulin antibody. In other examples, the ligand is an analyte or antigen to which the target antibody specifically binds.

In some examples, the target protein may be a tagged protein expressed in a genetically-engineered cell of interest. The tag may be, for example, a naturally-fluorescent protein fragment, such as green fluorescent protein (GFP), or an affinity-purification tag, such as a his-tagged heme-agglutinin (HA) fragment, which specifically binds both nickel and an anti-HA antibody, either of which may be conjugated or attached to the outer surface of the rapidly-sedimenting magnetic particle for isolation, detection, quantitation, processing, or characterization of the tagged protein.

In some examples, the target protein may be any other protein molecule of interest. The rapidly-sedimenting magnetic particle may include a ligand attached or conjugated to its outer surface. The ligand may be any molecule that specifically or selectively binds or interacts with the target protein molecule. The ligand may be, for example, any natural, synthetic, or genetically engineered antibody, either monoclonal or polyclonal including any fragment thereof, or any chemical or enzymatic modification of such an antibody. In other examples, the ligand may be a cell-surface receptor, a substrate or cofactor of the target protein, biotin, any biotin-specific ligand such as avidin or streptavidin, any antibody-specific molecule such as Protein A, a glycan-specific ligand that binds to certain glycoproteins, or any other member of a specific-binding pair of molecules.

In some examples, the rapidly-sedimenting magnetic particles of the present disclosure may be used to partially or completely purify the target protein from a sample solution. The protein may be, for example, a therapeutic reagent under production. In other examples, the magnetic particles may be used to selectively remove the target protein from the sample, where the protein may be poisonous, pathogenic, or in any other way dangerous if it remains in the sample.

### Isolation, Detection, Quantitation, Processing, or Characterization of Other Target Molecules or Objects in a Sample

Rapidly-sedimenting magnetic particles of the present disclosure may be used for the isolation, detection, quantitation, processing, or characterization of other target molecules or objects in a sample, using any of the methods described above. The target molecule or object may be, for example, a cell, subcellular organelle, exosome or other extracellular vesicle or particle, carbohydrate, lipid, metabolite, vitamin, cofactor, antigen, analyte, pathogen, bacterium, microbe, virus, poison, chemical, explosive, pollutant, dye, foodstuff, ink, paint, fabric, or the like. In some examples, the target molecule or object is an analyte, where the magnetic particles are used to isolate, detect, quantify, process, or otherwise characterize the analyte as part of a clinical or nonclinical assay for the presence and/or amount of the target analyte in a sample.

### Sample Types

Rapidly-sedimenting magnetic particles of the present disclosure may be used for the analysis, detection, quantitation, processing, or characterization of a target molecule or object in a variety of sample types.

In some examples, the sample may be a fluid from an organism, where the fluid may include blood, urine, sputum, spinal fluid, stool, mucus, ground or lysed organ or tissue, or any fraction thereof, including any cellular or subcellular fraction.

In some examples, the organism may be any biological organism, prokaryotic or eukaryotic, including any single-celled or multicellular plant, animal, protozoa, fungus, bacterium, or virus.

In some examples, the sample may be an in vitro solution containing the target molecule or object. The solution may further contain various salts or ions, pH buffers, detergents, chelating agents, blocking proteins, protease and/or nuclease inhibitors, and/or any other component suitable for the preservation, stability, or activity of the target molecule or object.

In some examples, the sample is a liquid biopsy sample. Rapidly-sedimenting magnetic particles of the present disclosure may be used, for example, for the faster and/or more efficient isolation and/or characterization of circulating pathogens, tumor-cells, or cell-free DNA in a blood sample, leading to faster and/or more sensitive diagnostic assays for these target molecules.

In some examples, the sample may be an environmental sample, such as soil, fauna, river water, ocean water, wastewater, drinking water, indoor air, outdoor air, or any other substance or object exposed to the environment, for the detection or characterization, for example, of target molecules that may be dangerous, poisonous, or in any other way hazardous to humans or to any other subject, object, or process. Rapidly-sedimenting magnetic particles of the present disclosure can be used, for example, for the faster and/or more efficient isolation of low-abundance microbes or cells in an environmental sample for metagenomic analysis. In other examples, the magnetic particles can be used for the faster and/or more efficient isolation, detection, or characterization of pollutants or contaminants in an environmental sample.

In some examples, the sample may be an industrial sample, to isolate, detect, quantitate, or in any other way characterize an industrial product, intermediate, by-product, or waste, in order to monitor the processing or production of an industrial product or to monitor any waste, contaminant, or pollutant generated by the processing or production of the product.

In some examples, the sample may be an agricultural sample or product to monitor or test the safety of the sample or product for use or consumption by a consumer.

In some examples, the sample may be a swab or swipe sample of a subject, such as a nasal or buccal swab, for the diagnosis of illness or disease in the subject.

In some examples, the sample may be a swab or swipe sample of an object for the detection of terrorism-related molecules, such as explosives or pathogenic molecules such as Anthrax.

In some examples, the sample may be a forensic sample for the isolation, detection, quantitation, processing, or any other characterization of a target molecule associated with evidence to a crime, such as for the identification of DNA or RNA in a dried semen or blood spot or stain recovered from a crime scene, a victim, or a suspect.

### Highly-Multiplexed Fluorescence Assays

Rapidly-sedimenting magnetic particles of the present disclosure may be used in multiplexed binding assays where different populations of magnetic particles are identifiable and differentiable based on a ratio of two or more labels, such as fluorescent dyes, on the particles. The different populations of magnetic particles may have different ligands conjugated or otherwise attached to the outer surface, so that each differentiable population of particles specifically binds and/or captures a different target molecule in a sample.

Rapidly-sedimenting magnetic particles of the present disclosure may be used in other fluorescent-dye-based binding assays, where the magnetic particles are used to bind, isolate, detect, quantify, move, mix, agitate, or in any other way process a target molecule or object, including the use of multiple dyes. The dye(s) may be attached to, or associated with, the magnetic particle, the target molecule, any magnetic-particle-associated or target-molecule-associated compound, or any combination thereof.

Similarly, rapidly-sedimenting magnetic particles of the present disclosure may be used in other label-based binding assays, where the label may be colored, radioactive, magnetic, spectroscopic, size-based, resonance-based, or any other suitable label.

### Lateral Flow Binding Assays

Rapidly-sedimenting magnetic particles of the present disclosure may be used in lateral-flow-type assays, where a labeled, first target-binding molecule in a mobile liquid-phase interacts with and/or binds to a target molecule in a sample, then migrates to a detection zone containing an immobilized second target-binding molecule. Capture of the target molecule bound to the first target-binding molecule by the second target-binding molecule as the bound (i.e., labeled) target molecule migrates through the detection zone localizes and concentrates the label at the detection zone. Detection of the label at the detection zone thereby detects the presence and/or quantity of the target molecule in the sample. The magnetic particles may be used to facilitate the movement or lateral flow of the mobile liquid-phase to the detection zone using applied magnetic fields or forces. In some examples, the labeled, first target-binding molecule is conjugated or otherwise attached to the magnetic particle. In other examples, the second target-binding molecule is conjugated or otherwise attached to the magnetic particles. In this way, binding of the target molecule to both the first and second target-binding molecules may occur in solution, providing for more efficient binding. The bound complex/sandwich of target molecule and target-binding molecules may then be drawn and immobilized to the detection zone using a magnet.

### Enzyme-Linked Immunosorbent Assays (ELISA)

In addition to lateral-flow-based assays, the rapidly-sedimenting magnetic particles of the present disclosure may be more generally used in an ELISA sandwich assay, where the binding of a labeled, first target-binding molecule and an immobilized, second target-binding molecule to the target molecule in a sample results in localization and/or concentration of the label at the site of the immobilized second target-binding molecule. Detection of the localized and/or concentrated label at that site thereby detects the presence and/or quantity of the target molecule in the sample. In some examples, the second target-binding molecule is immobilized to a fixed solid support, such as an internal surface of the sample vessel, as in traditional ELISA assays. The magnetic particles may be used to mix the solution to provide more efficient binding to the target molecule, particularly to the immobilized second target-binding molecule. In some examples, the second target-binding molecule may be reversibly immobilized to the sample vessel using magnetic forces. For example, the second target-binding molecule may be attached to the rapidly-sedimenting magnetic particle, allowing binding to the target molecule while the magnetic particle is suspended in solution. Such binding in solution is generally more efficient than the binding of the target molecule to the second target-binding molecule immobilized to a fixed solid substrate. The bound target molecule (i.e., sandwich of first target-binding molecule; target molecule; and second target-binding molecule) can then be pulled down to a detection zone in the sample vessel using magnetic forces. The rapidly-sedimenting magnetic particles may be pulled-down more quickly compared to traditional magnetic particles, thereby leading to a faster and/or more efficient or sensitive ELISA assay. In some examples, the magnetic particles may be pulled-down using a combination of magnetic and gravitational forces, (gravity-assisted magnetic isolation) as described previously, thereby allowing, for example, a rapid test/detection of trace material that may be present in a large sample volume.

The rapidly-sedimenting magnetic particles may also be used to mix the sample solution during binding to the target molecule, allowing a more efficient binding step. The mixing may be induced by applying a changing or alternating magnetic field, using, for example, an electromagnetic mixing device as described elsewhere in this disclosure. The mixing may be caused by the rapidly-sedimenting magnetic particles having the second target-binding molecule attached, or by a second population of magnetic particles included in or added to the sample solution.

### Cell Hashing

Rapidly-sedimenting magnetic particles of the present disclosure may be used in cell-hashing applications, where cell-surface-specific antibodies containing a barcoded oligonucleotide tag are used to differentiate nucleic acid samples from different individual cells. The magnetic particles may include, for example, a barcode-tagged antibody on its surface to capture or isolate a single cell of interest. Nucleic acid isolated from that cell may then be associated with that single cell in a pooled sample based on the barcode.

### Liquid-Phase Extraction

Rapidly-sedimenting magnetic particles of the present disclosure may be used for the isolation, detection, quantitation, processing, or characterization of a target molecule or object using liquid-phase extraction, where the target molecule or object has a preferential solubility or affinity for one of two or more immiscible fluids. In some examples, the immiscible fluids comprise an aqueous sample solution and a non-aqueous extracting solution, where the magnetic particles are predominantly soluble in one of the immiscible fluids. Target molecules in the sample solution may have a higher affinity for or solubility in the extracting non-aqueous solution, leading to extraction of the target molecules into the non-aqueous solution. Applied magnetic forces may then be used to help maintain the position of the magnetic-particle-containing phase, facilitating the separation of the sample-containing non-aqueous solution from the sample-depleted aqueous solution. In other examples, the target molecule may have a higher affinity for or solubility in the aqueous sample solution, while contaminants, impurities, or other non-target molecules may be extracted into the non-aqueous solution. Separation of the sample-enriched aqueous solution from the contaminant-containing non-aqueous solution, assisted by the use of the magnetic particles, leads to a more purified sample in the sample solution. In some examples, the rapidly-sedimenting magnetic particles may include a target-specific ligand for binding to the target molecule in one of the immiscible fluids. Examples of target-specific ligands can include one or more of proteinase k, a cocktail of nucleases, and a carboxyl group (or other ligand as described herein), such that incubation of a cellular sample with the enzyme-conjugated, carboxylated particles may lead to cell lysis, genomic DNA fragmentation, and selective binding to the DNA fragments, all in a single step. Magnetic forces can then be used to transfer or move the particle-bound target molecules into another one of the immiscible fluids to separate or isolate the target molecules from non-target molecules in the sample solution. In some examples, applied magnetic forces may be used to maintain or retain the bound target molecules in one of the immiscible fluids while the immiscible fluids are separated, and/or to isolate the target molecule from the fluid.

In some examples, at least one of the immiscible fluids is an ionic liquid. The ionic liquid may be reversibly immiscible in the other fluid, depending upon, for example, the temperature and/or pH of the ionic liquid or other fluid. In some examples, the target molecules are extracted into the ionic liquid and thereby separated from non-target molecules left behind in the other fluid. In other examples, non-target molecules are extracted into the ionic liquid, leaving behind a more purified sample in the other fluid. Rapidly-sedimenting magnetic particles of the present disclosure, which are soluble in one of the immiscible fluids, may be used to facilitate the extraction of target or non-target molecules into the ionic or other liquid.

In some examples, at least one of the immiscible fluids is a magnetic ionic liquid comprising rapidly-sedimenting magnetic particles of the present disclosure suspended in the liquid, where an ion, such as a carboxyl or amine group, attached otherwise bound to the surface of the particle, provides at least part of the ionic component of the ionic liquid. Typical magnetic ionic liquids of the prior art depend upon the easily hydrolyzed FeCl₄⁻ anion, which therefore may result in the release of iron into the solution, which may inhibit further downstream reactions, such as PCR. Rapidly-sedimenting magnetic particles of the present invention, particularly those including a coating encapsulating a magnetic core, may substantially reduce this release of iron into solution, making these particles more suitable for such iron-sensitive downstream reactions or processes. Ionic liquids comprising rapidly-sedimenting magnetic particles of the present disclosure may be used for the isolation of charged or polar molecules, such as DNA, from an aqueous sample solution, where the magnetic-particle-containing ionic liquid may easily switch from a water-soluble to a water insoluble form by a small change in, for example, temperature and/or pH.

### Bioreactor

Rapidly-sedimenting magnetic particles of the present disclosure may be used to support the growth of cells in a bioreactor, and/or to detect, quantitate, or differentiate cell-culture components in the bioreactor, such as cells, natural or engineered proteins produced in or secreted by the cells, metabolites, nutrients, salts, respiration-associated gasses, or the like.

In some examples, the rapidly-sedimenting magnetic particles are used to circulate, agitate, mix, or generally move the culture medium, by exposing the magnetic-particle containing medium to a changing magnetic field. In some examples, the changing magnetic field is applied by an electromagnetic mixing device as described elsewhere herein.

In some examples, the rapidly-sedimenting magnetic particles are used to detect and/or quantitate target molecules or objects in the medium related to the health of the cells or to a desired product produced by the cells. For example, the magnetic particles may include a target-specific ligand that binds to the target molecule or object of interest, or to a detectable label or some other detectable molecule or marker associated with the target molecule or object. Magnetic forces, in combination with gravitational forces, can then be used to draw or pull-down the magnetic particles onto predetermined detection zones, preferentially at the lowest point or points in the bioreactor. Since the rapidly-sedimenting magnetic particles have a natural tendency to settle to the lowest points in the bioreactor, the capture of the magnetic particles is facilitated, particularly for large-volume samples where the use of magnetic forces alone is impractical, as described for “gravity-assisted magnetic capture” elsewhere herein The captured target molecule, label, marker, or other detectable molecule may then be detected and/or quantified using any suitable in situ assay means. The assay may be performed within the general culture media, or in a separate chamber isolated from the culture media to avoid contamination of the cells. The captured magnetic particles may be moved into this chamber using applied magnetic forces. Alternatively, the captured molecules may be removed from the bioreactor through an outlet at the detection zone to allow off-board in vitro analysis of the captured molecule or object.

In some examples, the detection zone includes a photonic, electrochemical, or voltaic sensor or any other suitable sensor that detects and/or quantifies the captured target molecule or object in the detection zone.

In some examples, the target molecule may be a protein, such as a therapeutic antibody, secreted by the cultured cells. The magnetic particles may include a first ligand specific for the target protein, and the culture medium may contain a labeled, second ligand specific for the target protein.

In some examples, the target molecule may be a carbohydrate, fat, nucleic acid, co-factor, substrate, steroid, metabolite, or any other biological molecule associated with the cells in culture.

In some examples, the target object is a cell-type of interest in the culture, and the magnetic particle may include a ligand specific for a marker on the cell-type of interest, used for the quantitation, isolation, sorting, and/or analysis of the cell-type of interest.

In some examples, the target molecule is a detectable reagent or sensor, added to the culture media, indicative of the health of the cells.

In some examples, the bioreactor includes multiple detection zones, for the detection, quantitation, and/or analyses of multiple target molecules or objects.

### Acoustic Liquid Handling

In some examples, rapidly-sedimenting magnetic particles of the present disclosure are used in combination with an acoustic droplet ejection device. For example, magnetic forces may be used to move the magnetic particles to a predetermined vertical position in a well of a microtiter plate containing a liquid sample. Acoustic waves focused at this vertical position may then be used to acoustically eject droplets containing the magnetic particles, including any target molecule or object attached to or captured by the particle, thus avoiding the need to dynamically focus the acoustic wave onto the surface of the sample solution, which may be constantly changing as droplets are ejected.

Alternatively, acoustic forces may be used to move the rapidly-sedimenting magnetic particles in solution. For example, acoustic waves may be focused onto the magnetic particles bound to a target cell of interest via a target-cell-specific ligand attached to the particle, forcing the particle-bound target cells to the surface of the sample solution. The initial position of the magnetic particles bound to target cells may be determined, for example, by analyzing acoustic waves reflected from the sample (i.e., a miniaturized form of SONAR), as is common in this field. Acoustic waves focused onto the liquid surface May then be used to eject droplets selectively containing the cells of interest.

### Ball Milling

A ball mill uses the impact of tumbling metal balls in a spinning ‘hamster wheel’ device to break particles down to finer particles. In a similar manner, oscillating motion of the rapidly-sedimenting magnetic particles of the present disclosure may be used to break-up, grind, shear, pulverize, or otherwise disrupt a molecular or microscopic structure by essentially functioning as a microscopic version of a ball mill. The tumbling-like motion of the magnetic particles may be induced by applying an appropriate changing or oscillating magnetic field. The molecular or microscopic structure may include a biological tissue, a cell, a cellular organelle, a lipid membrane, a viral particle, a polynucleotide, a protein, a polysaccharide, chitin, or the like. By controlling the motion of the beads, the disruption May be relatively gentle, where, for example, lipid membranes are disrupted but covalent bonds are generally not broken, to more vigorous or severe, where covalent bonds in nucleic acids, polysaccharides, and the like, are broken.

The magnetic particles may be enclosed in a molecular cage to focus or localize the disruptive action of the magnetic particles.

### Tissue Disruption, Cell Lysis and Nucleic Acid Fragmentation

Induced motion of the rapidly-sedimenting magnetic particles of the present disclosure, by the application of a changing or oscillating magnetic field, may be used to break-up or disrupt biological tissues, cells, subcellular structures, organelles, nucleic acids, or the like. This disruption may be caused, for example, by thermal or kinetic energy of the particles, or by physical collision with the particles. In some examples, this disruption is augmented by the use of increased temperature and/or the addition of an enzymatic lysis reagent, such as proteinase k.

In other examples, the rapidly-sedimenting magnetic particles of the present disclosure may be used to disrupt, grind, or polish any other suitable non-biological material.

### Magnetic Resonance-Based Applications

Rapidly-sedimenting magnetic particles of the present disclosure can be used in various applications, including in medicine as biologically compatible and environmentally sensitive sensors and/or molecular imaging agents. For example, the magnetic particles can be used as magnetic resonance-based sensors in which the magnetic particles are used as remote sensors for detecting various analytes in an aqueous sample and can be used for the continuous monitoring of changing levels of analytes in the aqueous sample. The magnetic particles can be suspended or are suspendable in an aqueous liquid phase and be covalently or noncovalently linked to, or otherwise have immobilized thereon, one or more moieties selected to alter the state of aggregation of the magnetic particles as a function of the presence or concentration of the analyte in the solution.

In another example, rapidly-sedimenting magnetic particles of the present disclosure can be used in aggregate formation assays to detect target molecules. In the aggregate formation assays, a population of conjugates (or a mixture of two or more populations of conjugates with differing binding moieties directed to a target molecule or type of target molecule) is placed into a sample solution. Each conjugate comprises one or more binding moieties (e.g., an oligonucleotide, nucleic acid, polypeptide, or polysaccharide) linked, e.g., covalently or non-covalently, to a rapidly-sedimenting magnetic particle. The binding moiety causes a specific interaction with a target molecule (or, in some embodiments, an aggregation inducing molecule, such as avidin). The binding moiety specifically binds to a selected target molecule, which can be, for example, a nucleic acid, polypeptide, or polysaccharide. As a result, the dispersed state of the conjugates switches to an aggregated state, which decreases a spin-spin relaxation time (T2) of adjacent water protons in the aqueous solution. In some cases, the magnetic particles can simultaneously or in combination be used for magnetic separation of the target molecules.

In another example, the rapidly-sedimenting magnetic particles of the present disclosure can be used to detect target molecules in aggregate dispersion assays. In aggregate dispersion assays, conjugates are used to prepare small aggregates, and the aggregates are placed into a sample solution. In this assay system, the binding moieties are designed so that they can be bound to each other (or to a specific aggregation inducing molecule, such as avidin) to form the aggregates, and to be (or form upon binding to each other or to the aggregation inducing molecule) a substrate that is cleaved by a specific target molecule. If the sample solution contains a target molecule, the substrate formed by the binding moieties is cleaved, resulting in the dissolution of the aggregates. Thus, the aggregated state switches to a dispersed state, which increases T2 relaxation times.

The aggregates in these assay systems can be observed and detected in vitro, e.g., in vials, microplates, or solid substrates or arrays, as well as in vivo, e.g., using MR imaging of a subject after administration of the conjugates or aggregates. In some cases, the rapidly-sedimenting magnetic particles of the present disclosure can be used for imaging without requiring aggregation of multiple magnetic particles.

### Magnetic Hyperthermia Treatment

In another example, the rapidly-sedimenting magnetic particles of the present disclosure may be bound to target cells, such as cancer cells, for use in magnetic hyperthermia. In some examples, the magnetic particles may include a ligand that specifically binds to certain cancer cells. The magnetic particles may be provided to a cancerous cell or tissue, either in vitro or in vivo, so that the ligand-containing magnetic particles bind to the cancerous cell/tissue. In some examples, the magnetic particles may include a surface ligand that targets the magnetic particle to specific parts of a subject's body when injected systemically. In other examples, the magnetic particles may be directed to a cancer site in the subject using external magnetic forces.

Subsequently, an externally applied alternating magnetic field (e.g., 100 kHz) may be applied to the targeted magnetic particles such that the motion of the magnetic particles in response to the applied field generates an increase in thermal energy to treat the target cells/tissue. In particular, the increase in thermal energy may lead to destruction of the cancerous cells/tissue. Alternatively, the induced motion of the targeted magnetic particles may provide direct physical forces to disrupt tissue and/or cells at the cancer site.

### Cellular Transfection

In another example, the rapidly-sedimenting magnetic particles of the present disclosure can be used in cellular transfection, (magnetofection). In magnetofection, conjugated magnetic particles are bound to a target molecule, such as nucleic acid, and a magnetic field is then applied to the molecule bound magnetic particles to deliberately introduce and concentrate the particles into one or more target cells. The nucleic acids can then be released into the cell cytoplasm by various different mechanisms such as, for example: 1) the proton sponge effect, which is caused by cationic polymers coated on the magnetic particles that promote endosome osmotic swelling, disruption of the endosome membrane and intracellular release of the nucleic acid; or 2) the destabilization of the endosome by cationic lipids coated on the magnetic particle that release the nucleic acid into the cell by flip-flop of cell negative lipids and charge neutralization.

### Flow Cytometry and Cell Sorting

Rapidly-sedimenting magnetic particles of the present disclosure may be used in flow cytometry applications to facilitate the movement, focusing, detection, isolation, sorting, or processing of target cells or particles in a solution. For example, the magnetic particles may be used to generate a flow stream, in response to an applied magnetic field, to carry or propel target cells or particles through a viewing zone for analysis. In some examples, an applied magnetic field may be used to focus the magnetic particles into a narrow stream containing the cells or particles of interest. In some examples, the cells or particles of interest may be bound to a target-specific ligand on the magnetic particles, so that applied magnetic fields may be used to propel, focus, or otherwise direct the movement of the cells or particles of interest. In some examples, the magnetic particle may serve as a detectable label for target cells or molecules specifically bound to the magnetic particle, using a magnetic sensor. In some examples, directing the movement of the magnetic particles may include differentially sorting different target cells or particles bound to the magnetic particles using magnetic forces to direct the separate paths of the target cells or particles.

In some examples, the rapidly-sedimenting magnetic particles of the present disclosure may be used in a flow cytometry device comprising a magnetic emulsion analyzer using giant magnetoresistive sensors capable of multiparametric studies and sorting.

A combination of the physical and magnetic properties of the rapidly-sedimenting magnetic particles of the present disclosure of this disclosure may provide higher flow rates and/or more efficient detection, sorting, or analysis of target cells or particles using magnetic flow cytometry.

### Chemotherapy

Rapidly-sedimenting magnetic particles of the present disclosure may be used to deliver and target therapeutic molecules in a patient or subject. For example, the magnetic particles may be encapsulated in liposomes, emulsions, microgels, or other carriers containing a therapeutic compound. After introduction into a patient or subject, external magnetic forces may be used to direct the movement and/or targeting of the magnetic-particle containing carriers to a target site of interest, such as a tumor site. In some examples, the therapeutic compound may be conjugated to or otherwise attached to the magnetic particle. In some examples, a tumor-specific targeting molecule that specifically binds to a tumor-specific molecule on a tumor or cancer cell may be included in or on the magnetic-particle-containing carrier or conjugated or attached to the magnetic particle itself. In some examples, an external oscillating or changing magnetic field may be used to agitate the beads in order to disrupt tissue or to otherwise facilitate the movement of the magnetic beads in vivo. The disruption of tissue may be caused by the physical motion of the beads, or by heat generated by the induced movement of the beads. This disruption of tissue may be used for the targeted destruction of target cells of interest in vivo.

In some examples, the magnetic particles coupled to a therapeutic compound may be enclosed in a molecular ‘cage’, comprising covalent or other chemical bonds that may be broken once the magnetic particle has been localized to the tumor or cancer site, thereby localizing the release of the therapeutic to the tumor or cancer site. The covalent or other chemical bonds of the cage may be broken using light or any other electromagnetic energy, acoustic energy, ultrasonic energy, enzyme or other catalyst, or any other suitable means. In some examples, the therapeutic compound may be unattached or reversibly attached to the magnetic particles, such that breaking the covalent or other chemical bonds of the cage may release free therapeutic compound at the tumor or cancer site.

### Use of a Combination of Magnetic Particles

The rapidly-sedimenting magnetic particles of the present disclosure may be used in combination with any of the methods described above. For example, two or more different types of magnetic particle may be used together, where the different types of magnetic particle may differ in physical or magnetic properties, in the nature, identity, or structure of the ligand that may be attached to the surface of the particle, or in the function of the magnetic particle. In some examples, the first type of magnetic particle may have physical properties, such as size and/or density, that facilitate the mixing of the sample solution when subject to a changing external magnetic field, while the second type of magnetic particle may have chemical or biochemical properties that facilitate binding to a target molecule. A third magnetic particle may have physical and/or magnetic properties that lead to a faster magnetic capture of the beads using and external or inserted magnet. Any suitable number of different magnetic bead types may be used together.

In some examples, different types or species of ligand may be associated with a single magnetic particle, allowing multiple steps or processes to be performed on a sample in a single step. For example, a magnetic particle may contain both a protease, such as proteinase k, and a nucleic-acid-selective binding ligand, such as carboxyl, attached to its surface, allowing tissue digestion and/or cell lysis to occur via the protease, as well as isolation of the nucleic acids released by the digestion and/or lysis via the binding ligand. In another example, a magnetic particle may have multiple different enzymes attached to its surface, such as a cocktail of endonucleases having differing sequence-specificity, to allow cleavage of a target nucleic acid at multiple specific and/or non-specific restriction or digestion sites. In another example, a cocktail of different nuclease- and/or protease-inhibitors may be attached to a single magnetic particle, or to different particles used together in a sample, to protect target molecules in a cellular extract from digestion by endogenous enzymes. The inhibitor-coupled magnetic particles may then be easily removed from the sample using magnetic forces, allowing further processing of the target molecules without interference from the inhibitor cocktails. In further examples, one magnetic particle or subset of magnetic particles can include one ligand and a another magnetic particle or subset of magnetic particles can include a different ligand.

### Sequestration of a Reagent or Other Molecule

Rapidly-sedimenting magnetic particles of the present disclosure may be used to sequester a reagent, or any other molecule or object, in a reaction, either prior to or after its use in the reaction. For example, a reagent may be bound or attached to the magnetic particles, where the magnetic particles are sedimented or pulled-down to a side of a reaction vessel, before addition of a sample. This may effectively separate the reagent from the sample before the reaction is allowed to start, such as by increasing temperature. This may be used, for example, in a hot-start-like PCR reaction, where the polymerase may be sequestered from template and primer molecules to minimize non-specific amplification and primer dimer formation that may occur at room temperature while the reagents are being assembled. The polymerization reaction may be initiated at an elevated temperature while or after the magnetic particles are resuspend in the reaction solution. The beads may be resuspended by shaking, rocking, or otherwise agitating the sample vessel; by pipetting the reaction solution up and down; by stirring the reaction solution, or by applying an oscillating, alternating, or otherwise changing external magnetic field.

In some examples, the reagent may be sequestered from other components in the reaction as an alternative to physically, chemically, or enzymatically inactivating the reagent.

Sequestration of the reagent may be facilitated by the natural tendency of the rapidly-sedimenting magnetic particles of the present disclosure to settle to and remain at the bottom of a container, so that the particles may remain sequestered there even after the magnetic force used to pull them down has been removed.

### Ion Binding or Exchange

The rapidly-sedimenting magnetic particles of the present disclosure mat be used in various ionic-binding or ion-exchange applications. For example, the magnetic particles may include a chelating agent attached to the surface of the particle, allowing the beads to be used to selectively bind to certain ions in a sample solution, such as cationic metal ions. These particles may be used, for example, to bind and remove interfering metal-ions from a sample prepared for mass spectrometry analysis. In other examples, the magnetic particles may be used to detect and/or quantify metal contaminants in a sample, such as water released from a nuclear power plant, or to detect and/or quantify toxic rare metals that may be present in a biological sample.

The rapidly-sedimenting magnetic particles of the present disclosure may be used in various ion-exchange applications. Magnetic particles having a negative charge, such as particles having a carboxyl group exposed on the outer surface, may be used for cation exchange chromatography or other cation-exchange applications. In some examples, the negatively-charged magnetic particles may be used to isolate or purify drugs during microsynthesis, or to isolate, test, or identify phytochemicals as potential therapeutics. In some examples, the negatively-charged magnetic particles may be used to remove inhibitory cations in a sample solution, such as Mn⁺² or Fe⁺³, for example, that may inhibit an enzyme used in a reaction, such as a polymerase used in a PCR reaction, or any other susceptible enzyme used in a reaction or assay.

Negatively-charged, rapidly-sedimenting magnetic particles of the present disclosure may be used as the solid phase, support, or substrate for cationic exchange of cationic proteins, or any other cationic molecule of interest. Cationic proteins, for example, may be isolated from a sample by allowing binding of the proteins to the magnetic particles, and then separating and isolating the protein-bound beads from the sample solution. The proteins may be eluted from the isolated particles by resuspending the particles in a buffer at an appropriate pH. In some examples, the negatively-charged magnetic particles selectively bind to a polyhistidine tagged protein, providing an alternative to the potentially toxic nickel-based approaches for the isolation of such polyhistidine-tagged proteins.

Negatively-charged, rapidly-sedimenting magnetic particles of the present disclosure may be used to remove a cationic detergent, such as a benzalkonium, a tetraalkylammonium, an alkylpyridinium, a dialkylpyrrolidinium, a polyethyleneimine, or any other cationic detergent, from a sample solution.

The rapidly-sedimenting magnetic particles of the present disclosure may include a positively-charged/cationic group or moiety, such as amine, attached to the outer surface. Such particles may be used, for example, in various anionic-exchange applications. In some examples, the particles may be used for the removal of an anionic detergent, such as SDS, alkylsulfonates, alkylbenzene sulfonates, bile acids, etc., from a sample solution. In other examples, the particles may be used for removal of polyanionic ions, such as pyrophosphate, phytic acid, inositol phosphates, nucleotides, nucleic acids, polygalacturonic acids, and the like. In some examples, the particles may be used for the isolation/purification of an anionic protein, where elution of the anionic protein from the particle may be accomplished by a change in pH.

### FRET Assays Using Magnetic Particles

The rapidly-sedimenting magnetic particles of the present disclosure may be used in various assays that rely on fluorescence resonant-energy transfer (FRET). For example, an analyte of interest may be labeled with a first fluorescent dye. This labeling may be accomplished, for example, by allowing the binding of the analyte to an analyte-specific binding molecule including the first fluorescent dye, or by any other suitable means. The magnetic particle may include a second analyte-specific binding molecule, as well as a second fluorescent dye that is excited by the emission of the first dye and emits light of a wavelength higher/longer than that of the first dye, but only when the first and second dyes are in close physical proximity, due to binding of the labeled analyte to the magnetic particle. In this way, detected fluorescence of the second dye (i.e., at the higher/longer wavelength) is indicative of binding of the analyte to the magnetic particle (and therefore the presence of the analyte in the sample). In some examples, the binding of the analyte to the magnetic particles occurs in solution. The binding may be facilitated by the mixing of the analyte-containing solution during binding. This mixing may be accomplished, for example, by applying a changing external magnetic field, or by any other suitable means. In some examples, the fluorescence of the second dye is measured or detected as overall fluorescence from the sample solution, as is common in homogeneous assays. In other examples, the analyte-bound magnetic particles may be pulled-down using magnetic forces to concentrate the particles and their associated FRET signal to a localized detection zone, thereby increasing the detection signal. In this way, the signal obtained from a typical homogeneous assay (based on FRET) is amplified by physically concentrating the analyte of interest.

### A. Particles

As used herein, “ferrimagnetic particles” refers to rapidly-sedimenting magnetic particles comprising a ferrimagnetic material. Ferrimagnetic particles can respond to an external magnetic field (e.g., a changing magnetic field), but can demagnetize when the external magnetic field is removed. Thus, the ferrimagnetic particles are efficiently mixed through a sample manually (e.g., mixing by hand or though aspiration using a pipette) or by external magnetic fields as well as efficiently separated from a sample using a magnet or electromagnet, but can remain suspended without magnetically induced aggregation occurring.

The ferrimagnetic particles described herein are sufficiently responsive to magnetic fields. In general, the range of the field intensity could be the same range as any electromagnet as long as it is able to move the particles. For example, the magnetic field can have an intensity in a range of from about 10 mT and about 100 mT, about 20 mT and about 80 mT, and about 30 mT and about 50 mT. In some examples, more powerful magnets can be used to mix less responsive particles. In some examples, the magnetic field can be focused into the sample as much as possible. Also, the electromagnets can be as close to the sample as possible since the strength of the magnetic field can decrease as the square of the distance.

In some examples, the ferrimagnetic particle includes a core that includes a ferrimagnetic material. An example of a ferrimagnetic material is a ferrite. A ferrite includes a ceramic material that includes an oxide of iron in combination with inorganic compounds of metal, non-metal, or metalloid atoms. For example, a ferrite can include iron (III) oxide (Fe₂O₃) blended with one or more additional metallic elements, such as barium, manganese, nickel, zinc, titanium, or any other suitable metallic element. Other examples of ferrites include Fe₂TiO₂, FeTiO₂, MnFe₂O₄, NiFe₂O₄, MgFe₂O₄. Further examples of ferrites include an iron core including a sulfide or an oxyhydroxide such as Fe₇S₈, Fe₃S₄, FeS, or FeOOH.

Magnetite (Fe₃O₄) is another example of a ferrimagnetic material useful in the examples described herein that is an example of a ferrite. Magnetite contains both Fe²⁺ and Fe³⁺ ions. In some cases, the electron spins of the Fe²⁺ and Fe³⁺ ions can be coupled in a crystalline structure such that the magnetite is ferrimagnetic, as described herein. However, in some examples, ferrimagnetic particles comprise any ferrimagnetic material (e.g., ferrite). According to some examples, the ferrimagnetic material (e.g., ferrite) may not be magnetite (Fe₃O₄), however in some examples, magnetite is a suitable ferrimagnetic material.

Ferrites can be categorized into two main families (hard ferrite and soft ferrites) based on their magnetic coercivity (e.g., the material's ability to withstand an external magnetic field without becoming demagnetized).

Hard ferrites have a high magnetic coercivity as well as a high remanence after magnetization. Hard ferrites can be used to make permanent magnets, as hard ferrites do not demagnetize easily in the absence of an external magnetic field, as they can have a high remanence. Examples of hard ferrites include strontium ferrite and barium ferrite.

Soft ferrites have a low magnetic coercivity. Soft ferrites also have a low remanence after magnetization. The magnetization of soft ferrites is easier to change than hard ferrites. Further, the magnetization of soft ferrites can easily reverse direction without dissipating large amounts of energy (e.g., via hysteresis losses). Soft ferrites can also have a high electrical resistivity, thus preventing the formation of eddy currents in the material, which is another source of energy loss.

Soft ferrites can include manganese-zinc (MnZn) ferrite and nickel-zinc (NiZn) ferrite. Thus, in some examples, the ferrimagnetic particles comprise MnZn ferrite. In other examples, the ferrimagnetic particles comprise NiZn ferrite. Ferrimagnetic particles comprising MnZn ferrite and/or NiZn ferrite can become magnetized in the presence of an external magnetic field, and thus are able to be moved in the presence of the external magnetic field, but do not aggregate due to magnetically induced aggregation after the external magnetic field is removed, since they have a low remanence.

Some ferrites can be considered to be semi-hard ferrites. Semi-hard ferrites have properties that are between the properties of soft ferrites and the properties of hard ferrites. For example, cobalt ferrite (CoFe₂O₄) is a semi-hard ferrite, which can be magnetized in the presence of an external magnetic field (e.g., a changing magnetic field generated by a magnetic assembly), but does not have a high remanence after the external magnetic field is removed, such that the ferrimagnetic particles comprising a cobalt ferrite core do not aggregate due to magnetically induced aggregation.

A “magnetic domain” is a region within a magnetic material in which the net magnetization is in a uniform direction. Magnetic domains can occur in ferromagnetic and ferrimagnetic materials. A material can include many magnetic domains. The magnetization within a magnetic domain can point in a uniform direction. Each magnetic domain in a material can be oriented in a different direction. In the presence of an external magnetic field, the domains in a magnetic material can rotate so that each domain's magnetization aligns with the external magnetic field.

The term “remanence” refers to residual magnetism that a material retains after a magnetic field has been removed. Materials that have a high remanence after the magnetic field has been removed retain a large magnetic field strength, whereas materials that have a low remanence after the magnetic field has been removed have a small magnetic field strength or zero magnetic field strength.

Those of skill recognize a number of different types of magnetism including paramagnetism, superparamagnetism, ferromagnetism, antiferromagnetism, and ferrimagnetism. Paramagnetism occurs in the presence of unpaired electrons in a material. The magnetic moments in a paramagnetic material is not aligned and can point in random directions due to thermal motion. The material has a net magnetism of zero since the magnetic moments point in random directions, thus cancelling one another out. In the presence of an external magnetic field, the magnetic moments align parallel to the external magnetic field. The paramagnetic material then forms an induced magnetic field in the direction of the external magnetic field, causing a net attraction. Paramagnetic materials only exhibit magnetism in the presence of an external magnetic field. Paramagnetic materials can be weakly magnetically responsive. Examples of paramagnetic materials include aluminum, oxygen, titanium, and iron oxide (FeO).

Materials that are ferromagnetic can be magnetized by an external magnetic field, e.g., the magnetic moments of the material align in the same direction, and remain magnetized after the external magnetic field is removed. A ferromagnetic material can form an induced magnetic field in the direction of the aligned magnetic moments.

Ferromagnetism is a property not just of the chemical make-up of a material, but also of the material's crystalline structure and microstructure. For example, there are ferromagnetic metal alloys that comprise elements that are not ferromagnetic. A ferromagnetic material has a high susceptibility to an external magnetic field and tends to retain a magnetic field after the external magnetic field is removed. Particles comprising a ferromagnetic material can undergo magnetically induced aggregation since they retain a magnetic field. Thus, after a magnetic mixer mixes ferromagnetic particles throughout a sample, the ferromagnetic particles can remain magnetized and clump together. Examples of ferromagnetic materials include iron, nickel, and cobalt.

A ferrimagnetic material can have multiple populations of atoms with opposing magnetic moments. The magnetic moments of one population can be stronger than the magnetic moments of another population, thus causing a net magnetism. The crystal structure of a ferrimagnetic material comprise magnetic sublattices of magnetic moments, wherein the magnetic moments of the two sublattices are anti-aligned and not equal. The opposing magnetic moments are unequal and a spontaneous magnetization remains. Ferrimagnetic materials can also have a high electrical resistivity. When the external magnetic field is removed from a ferrimagnetic material, the ferrimagnetic material can remain magnetized or can become unmagnetized depending upon the specific ferrimagnetic material. An example of a ferrimagnetic material is a ferrite.

Superparamagnetism is another type of magnetic behavior, in which nanoparticles, for example smaller than 50 nm in size, made of a ferromagnetic or ferrimagnetic material, are small enough to contain a single magnetic domain. Superparamagnetic materials can exhibit paramagnetic-like behavior outside of a magnetic field, but can be more magnetically responsive than paramagnetic materials in the presence of an external magnetic field.

According to various examples, magnetic strength of the ferrimagnetic particles can be greater than or equal to about 20 emu/g, about 25 emu/g, about 30 emu/g, about 35 emu/g, about 40 emu/g, about 45 emu/g, about 50 emu/g, about 75 emu/g, about 100 emu/g, about 150 emu/g, about 175 emu/g, about 200 emu/g, about 225 emu/g, about 250 emu/g, in a range of from about 20 emu/g to about 250 emu/g, or about 35 emu/g to about 100 emu/g. This value can be considered to be the maximum field strength of the particle, which is a measure of the magnetic strength generated by the particle upon exposure to a magnetic field. In combination with the magnetic strength of the ferrimagnetic particles, the permeability of the ferrimagnetic particle should be sufficient to generate an induced magnetic field greater than or equal to about 1 emu/g, 5 emu/g, 10 emu/g, 15 emu/g, 20 emu/g, about 25 emu/g, about 30 emu/g, about 35 emu/g, about 40 emu/g, about 45 emu/g, about 50 emu/g, about 75 emu/g, about 100 emu/g, about 150 emu/g, about 175 emu/g, about 200 emu/g, about 225 emu/g, about 250 emu/g, in a range of from about 10 emu/g to about 250 emu/g, or about 35 emu/g to about 100 emu/g. The magnetic field to which the ferrimagnetic particles are exposed, can have a strength of about 700 Oersted to about 800 Oersted, about 725 Oersted to about 775 Oersted, less than, equal to, or greater than about 700 Oersted, 725, 750, 775, or about 800 Oersted.

According to various examples, the remanence of the ferrimagnetic materials can be in a range of from about 0 emu/g to about 30 emu/g, about 0 emu/g to about 10 emu/g, about 1 emu/g to about 8 emu/g, about 3 emu/g to about 5 emu/g, less than, equal to, or greater than about 0 emu/g, 1, 2, 3, 4, 5, 6, 7, 8, 9, 10, 11, 12, 13, 14, 15, 16, 17, 18, 19, 20, 21, 22, 23, 24, 25, 26, 27, 28, 29, or about 30 emu/g.

The ferrimagnetic particles can be a variety of shapes, which can be regular or irregular. The shape of the ferrimagnetic particles can largely be a function of the shape of the ferrimagnetic core. In some examples, the shape maximizes the surface areas of the particles. For example, the ferrimagnetic particles can be spherical, bar shaped, elliptical, or any other suitable shape. The ferrimagnetic particles can be a variety of densities, which can be determined, at least in part, by the size and composition of the core. In some examples, the density of the ferrimagnetic particles can be adjusted with a coating, as described herein.

In some examples, the ferrimagnetic particles have sufficient surface area to permit efficient binding of a target analyte and are further characterized by having surfaces which are capable of reversibly or irreversibly binding the target analyte (e.g., biological molecules). In some examples, a surface area of the ferrimagnetic particles can be in a range of from about 0.1 m²/g to about 500 m²/g, about 50 m²/g to about 200 m²/g, or about 150 m²/g to about 175 m²/g.

Suitable ferrimagnetic particles can be of a size that their separation from solution is not difficult, for example by magnetic means or by filtration. In addition, ferrimagnetic particles should not be so large that their surface area is minimized or that they are not suitable for nanoscale to microscale manipulation

Suitable major dimensions (e.g., a mean diameter) ranges from about 1 nm mean diameter to about 1 mm mean diameter, about 5 nm to about 50 μm, or about 75 nm and about 100 μm, about 80 nm to about 300 nm, about 80 nm to about 200 nm, about 95 nm to about 150 nm. For example, in some examples, the ferrimagnetic particles can be nanoparticles (e.g., particles having a mean diameter less than 1 μm, but greater than 1 nm). In other examples, the ferrimagnetic particles can be microparticles (e.g., particles having a mean diameter greater than 1 μm, but less than 100 μm). In general, larger ferrimagnetic particles (that is about 1 mm in size) are useful in cellular fractionation, tissue digestion, liquid mixing, and the like.

The ferrimagnetic particles can be substantially solid or can have some degree of porosity. Where the ferrimagnetic particles do include some degree of porosity, a pore size of the individual pores can be in a range of from about 0.5 nm to about 100 nm, about 5 nm to about 50 nm. At least a plurality of the pores can be through pores (e.g., extending fully between opposed surfaces). The pore sizes or total porosity of the ferrimagnetic particles can be determined according to many suitable methods. For example, the bulk volume of an ideal (e.g., non-porous) ferrimagnetic particle can be determined and then the volume of the actual porous skeletal material can be determined. The porosity is then calculated by subtracting the volume of the actual porous skeletal material from the ideal ferrimagnetic particle. The porosity of the ferrimagnetic particle or individual pore size can also be determined through optical measurements using a microscope and processing the images to measure the individual pores.

The ferrimagnetic particles described herein can include several different materials. To the extent that mixtures of materials are present, the total magnetic content of the ferrimagnetic particles can constitute at least 50 wt % of the ferrimagnetic particle, at least 70 wt % of the ferrimagnetic particle, or even 100 wt % of the ferrimagnetic particle. The ferrimagnetic particles can include any of those described herein. The non-magnetic material constituting the balance of the ferrimagnetic particles can include any of the coating materials described herein, for example. Non-magnetic material can be used as a coating to encapsulate the magnetic portion of the ferrimagnetic particle, they can also be used as a functional component to interact with and bind an analyte of interest. Non-magnetic material can also act a as filler component.

The magnetic components of the particles can be magnetic nanoparticles, magnetic sub-micrometer particles, or magnetic micrometer particles. The ferrimagnetic particles described herein can have many different structures. For example, the ferrimagnetic particles can be magnetic nanoparticles incorporated in a silica or polymer matrix, magnetic beads encapsulated in a silica or polymer shell, magnetic nanoparticles or functionalized magnetic beads, bare magnetic nanoparticles or beads. In examples where the ferrimagnetic particles are core-shell particles, the shell can include a coating as described herein.

### B. Coating

The ferrimagnetic particles of the disclosure can include a ferrimagnetic core or inner layer, partially or fully surrounded by one or more coatings. In an example, the ferrimagnetic particles are coated with one or more layers of a non-magnetic material. The use of coated ferrimagnetic particles, having no exposed iron, on their surfaces, can substantially eliminate the possibility of iron interfering with certain downstream manipulations of the sample.

According to various examples, the rapidly-sedimenting magnetic particle can include a coating layer. The coating layer partially or fully coats the magnetic material. The coating layer may be an internal layer or an outer coating. The coating layer can include, for example, a polymer layer, or a silica layer. The thickness of the core coating layer can have an average or absolute value ranging from about 1 nm to about 100 nm.

When the coating layer includes a polymer, examples of polymers can include polyethylene, polystyrene, poly methyl methacrylate, polyvinyl alcohol, or any other suitable polymer. When the coating layer includes a silica, examples of silica can include silicon dioxide, borosilicate, soda lime, barium titanate, and other types of glass. The thickness and composition of the coating layer can help to adjust the density of the ferrimagnetic particles to be close to the density of the sample to which the rapidly-sedimenting magnetic particles are added for analysis, for example, an aqueous sample (e.g., approximately 1 g/cm³). The thickness and composition of the coating layer may also be adjusted to increase the overall density of the ferrimagnetic particle to increase its sedimentation rate. In other examples, the ferrimagnetic particles may include an additional or alternative layer, whose composition and thickness may be adjusted to modify the overall density of the particle and thereby its sedimentation rate.

In other examples, other types of materials for the coating layer can include metal plating such as aluminum, gold, zinc oxide, or any of the other coatings mentioned herein, etc. Furthermore, any of the coatings described herein can have a fluorescent or colored dye included.

The outer coating layer can include a free functional group, including those mentioned herein, for selectively or non-selectively binding target analytes. The functional group can be adapted for adsorbing biomolecules, such as nucleic acids, which can non-sequence-specifically and reversibly bind to the functional group coating the ferrimagnetic particles. The polynucleotides can be DNA, RNA, or polypeptide nucleic acids (PNAs).

In an example, the functional group is a free carboxyl group. A suitable moiety with a free carboxylic acid functional group is a succinic acid moiety in which one of the carboxylic acid groups is bonded to the amine of amino silanes through an amide bond and the second carboxylic acid is unbonded, resulting in a free carboxylic acid group attached or tethered to the surface of the ferrimagnetic particle. In some aspects, the free carboxylic acid is a terminal group of a polymer tethered to the core coating layer. The tethered polymer can be poly(methyl vinyl ether-alt-maleic anhydride) with the carboxyl group tethered thereto. The poly(methyl vinyl ether-alt-maleic anhydride) can have a weight-average molecular weight in a range of from about 200 kDa to about 500 kDa, about 250 kDa, less than, equal to, or greater than about 200 kDa, 210, 220, 230, 240, 250, 260, 270, 280, 290, 300, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or about 500 kDa.

Other suitable functional groups that can used in the outer layer coating include, but are not limited to thiol groups, streptavidin, avidin, neutravidin, captavidin, amine groups, hydroxyl groups, tosyl groups, epoxy groups, alkyl groups, vinyl groups, or aryl groups. According to further examples, the surface can be coated with a biomolecule, such as an enzyme, protein, deoxyribonucleic acid (DNA), ribonucleic acid (RNA), immunoglobulin G, immunoglobulin A, or an antibody (e.g., a monoclonal antibody).

Various coatings comprising functional groups suitable for these purposes are described in U.S. Pat. Nos. 5,705,628, 5,898,071, and 6,534,262, the teachings of which are hereby incorporated by reference into this application in their entirety. Any of the coatings described herein can be functionalized with surface chemicals as described herein, for example, with carbolic acid, streptavidin, amine, hydrazide, silanol, azide. And those can be further functionalized with biological molecules such as antibodies, enzymes, DNA or RNA fragments, catalysts, etc.

In some examples, the outer coating layer can include a capture reagent. The capture reagent can be for capturing an analyte in a sample. The surface of the ferrimagnetic particles can be coated with a capture reagent (e.g., antibodies, lectins, oligonucleotides, other affinity groups, or any of the other capture reagents mentioned herein), which can selectively bind a target analyte or a group of analytes in a mixture. In some examples, the capture reagent can be an antibody.

Those of skill will recognize that any number of capture reagents can be used for this purpose, e.g., aptamers, nanoparticles, binding proteins, and the like. The capture reagent can be designed to capture a specific analyte or a specific panel of analytes, e.g., drug panel or endocrine panel, etc.

Alternatively, the capture reagent can include an enzyme. In some embodiments the enzyme can be linked to the coating in order to selectively interact with a substrate of that enzyme. Upon interacting with the substrate, the enzyme can function to degrade or digest the substrate. This can lead to generation of a substance of interest through enzyme's action or to remove a substrate from a sample. According to various embodiments, the enzyme can be trypsin. In some examples, the enzyme can be a nucleic acid fragmentation enzyme. An example of a nucleic acid fragmentation enzyme includes a caspase-activated DNase, a fragmentase, a micrococcal nuclease, or a mixture thereof. According to various further embodiments, the enzyme can be a lysing enzyme. An example of a lysing enzyme can include a lysozyme, a proteinase K, a collagenase, or a mixture thereof.

In some examples, the outer coating layer can include a mixture of any of the components described herein. For example, in some aspects the rapidly-sedimenting magnetic particle can include both enzymes and free carboxyl groups in the outer coating layer. As a further example, the outer coating layer can include two different enzymes. In still further examples, the outer coating layer can include two different free carboxyl groups.

To help bond the core coating layer and the outer coating layer, the rapidly-sedimenting magnetic particle can include a linking layer disposed therebetween. The linking layer can include any suitable material to facilitate a bond between the core coating layer and the outer layer. An example of such a material includes a silane linker. An example of a suitable silane linker includes N-(3-triethoxysilylpropyl) gluconamide.

A sample used in the present disclosure can be a fluid sample and can be, for example, a biological sample or a chemical sample. As used herein, “biological samples” can include biological fluids and may include, but are not limited to, blood, plasma, serum, or other bodily fluids or excretions, such as but not limited to saliva, urine, cerebrospinal fluid, lacrimal fluid, perspiration, gastrointestinal fluid, amniotic fluid, mucosal fluid, pleural fluid, sebaceous oil, exhaled breath, and the like. Chemical samples can include any suitable types of samples comprising chemicals to be detected, including water samples.

Appropriate biological samples may also include lysates prepared from cells obtained from either mammalian tissue, cell culture, or body fluids, nucleic acid samples eluted from agarose or polyacrylamide gels, solutions including deoxyribonucleic acid (DNA) molecules or ribonucleic acid (RNA) molecules, solutions containing multiple species of DNA molecules resulting either from a polymerase chain reaction (PCR) amplification or from a DNA size selection procedure and solutions resulting from a post-sequencing reaction. Suitable samples can be mixtures of biomolecules (e.g., proteins, polysaccharides, lipids, low molecular weight enzyme inhibitors, oligonucleotides, primers, templates, nucleic acids) and other substances such as agarose, polyacrylamide, trace metals and organic solvents, from which the target nucleic acid molecule can be isolated.

The term “analyte” refers to a substance whose presence, absence, or concentration is to be determined according to examples of the present disclosure. Examples of analytes may include, but are not limited to any of the biological sampled described above. Analytes can also include specific molecules, such as DNA, RNA, hormones (such as thyroid hormones, estradiol, testosterone, progesterone, estrogen), metabolites (such as glucose or ethanol), proteins, lipids, carbohydrates and sugars, steroids (such as Vitamin D), peptides (such as procalcitonin), and nucleic acids. The analyte can also be cells, cell components (such as cell membranes), spores, biomarkers (pharmaceuticals such as antibiotics, benzodiazepine), drugs (such as immunosuppressant drugs, narcotics, opioids, etc.), molecules with a regulatory effect in enzymatic processes such as promoters, activators, inhibitors, or cofactors, microorganisms, such as viruses (including EBV, HPV, HIV, HCV, HBV, influenza, norovirus, rotavirus, adenovirus (e.g., adeno-associated virus), coronavirus that causes severe acute respiratory syndrome (COVID-19), etc.), bacteria (Helicobacter pylori, Streptococcus, methicillin-resistant Staphylococcus aureus, Clostridium difficile., Legionella, etc.), fungi, parasites (plasmodium, etc.). Examples of the disclosure can also allow for the simultaneous analysis of multiple analytes in the same class or different classes (e.g., simultaneous analysis of metabolites and proteins). In examples of the disclosure, the analysis of a particular analyte such as a biomarker may indicate that a particular condition (e.g., disease) is associated with a sample that contains the analyte.

In some specific examples, an analyte of interest can include viral RNA. Specifically, an analyte of interest can include RNA from a corona virus. More specifically, the analyte of interest can include RNA from a coronavirus that causes severe acute respiratory syndrome (COVID-19).

### C. Manufacture

The ferrimagnetic particles can be manufactured using any suitable method of manufacturing nanoscale to microscale rapidly-sedimenting magnetic particles. As examples, U.S. Pat. No. 5,648,124 and PCT publication WO 2020/018919 each discloses a process for preparing magnetically responsive microparticles, and are hereby incorporated by reference herein in their entirety. The ferrimagnetic particles can be manufactured using any suitable ferrimagnetic material, as described herein.

For example, a ferrimagnetic particle can be manufactured by first adding ferrimagnetic nanoparticles to a chemical bath. The nanoparticles can be encapsulated in an inorganic silica matrix, thus producing a microparticle that contains many ferrimagnetic particles. Sonication can then be used to help produce these particles in a monodispersed fashion. Although a silica matrix is mentioned above, it is also possible for individual ferrimagnetic nanoparticles or microparticles to be encapsulated in other inorganic or organic materials. For example, the ferrimagnetic nanoparticles can be encapsulated in SiO₂, TiO₂, ZnO₂, Al₂O₃, CeO₂, or any suitable ceramic material. As a further example, the ferrimagnetic nanoparticles can be encapsulated in an organic material such as polyacrylic acid (PAA), poly(methyl acrylate) (PMA), polystyrene (PS), divinylbenzene (DVB), polyvinylpyrrolidone (PVP), or polyvinyl alcohol (PVA).

In another example, a ferromagnetic material can be used to manufacture ferrimagnetic particles. The magnetic properties can be altered by changing the structure of the ferromagnetic material. Hematite (Fe₂O₃) is naturally ferromagnetic when allowed to crystalize in its pure form. However, if impurities like nickel and zinc are added, then the nickel and zinc can take the place of some of the iron in the crystalline structure, thus turning the naturally ferromagnetic material into a ferrimagnetic particle. Or, in a different example, ferromagnetic hematite can be ground down to less than 50 nm in size such that each particle contains a single magnetic domain. In this form, the particle can be a superparamagnetic particle. An exemplary ferrimagnetic particle can be made from ferrimagnetic magnetite nanoparticles 50-100 nm in size joined together in silica or polymer. These nanoparticles are too large to be superparamagnetic.

The present teachings generally relate to sample processing methods and systems for mixing, separating, filtering, or otherwise processing a sample (e.g., a fluid sample) in a container by utilizing ferrimagnetic particles of the disclosure that are caused to move under the influence of a magnetic assembly disposed about or below a container that the rapidly-sedimenting magnetic particles are disposed within.

According to various embodiments, a sample processing system of the disclosure can include a pipette or an array of pipettes, a container or an array of containers, ferrimagnetic particles, an optional magnetic assembly reversibly disposed about the periphery of at least one container, a magnetetic assembly reversibly positioned below at least one container and/or a control component coupled to the magnetic assembly. Where present, the magnetic assembly can be reversibly disposed about the container can include at least one magnetic structure, each magnetic structure can include a plurality of electromagnets disposed about the periphery of the container. Each electromagnet can be individually controlled by the control component to generate a desired magnetic field within the container effective to influence the ferrimagnetic particles, for example, in accordance with a sample processing method comprising various steps. In some examples, the magnetic assembly can include a plurality of magnetic structures. The magnetic structures can be arranged in horizontal or substantially horizontal layers. In other examples, the magnetic structures can be arranged in vertical or substantially vertical layers.

In yet other examples, there can be a magnetically-permeable field shorting plate or structure below, above, and/or between the magnetic structures. The magnetically-permeable field shorting plate drastically reduces power consumption by concentrating the magnetic field in one particular location instead of two.

In some examples, the sample processing system can include a magnet (e.g., a permanent magnet) or an electromagnet capable of collecting the ferrimagnetic particles in the container at a desired site in the container, thereby allowing the ferrimagnetic particles to be separated from the sample disposed in the container. For example, the magnet can be removably positioned beneath a container that includes the sample. The magnet can be removably positioned below the sample in that the magnet itself can be moved relative to the container or the container can be moved relative to the magnet.

The sample can be placed in the container by a pipette (or a plurality of pipettes). The pipette can be automated with its movements dictated by a controller. In some embodiments, the pipette, or an array of pipettes, can be controlled in such a manner that the pipettes can collect the sample and dispense it into the container. The pipettes can further be controlled in such a manner that they can collect buffers, reagents, the rapidly-sedimenting magnetic particles or any other material needed and place them in the container. In some embodiments, the pipettes can further be controlled to mix the components in the container by aspirating the components.

It will be appreciated by those skilled in the art that the container, magnetic assembly, and the control component can be configured in any suitable manner to generate changing magnetic fields (e.g., oscillating magnetic fields, rotating magnetic fields) in the container. PCT Application No. PCT/IB2016/057189 to Arnold et al. discloses electromagnetic assemblies for processing fluids suitable for use in the present disclosure, and is hereby incorporated by reference herein in its entirety.

In operation a method of using the system can include supplying the rapidly-sedimenting magnetic particles, reagents, and biological sample to the one or more containers. The components placed in the containers can be supplied by one or more automated pipettes. Once the containers are loaded with the components, the containers can be exposed to a magnetic field. To expose the containers to the magnetic field, the containers can be brought near to a permanent magnet or the permanent magnet can be brought near the containers. While the permanent magnet can be positioned in many different ways, at least one permanent magnet can be positioned underneath the containers.

The components in each container can be mixed. Mixing can by any suitable method. For example, mixing can be accomplished by hand. As a further example, mixing can be accomplished through aspiration using the pipette. Following mixing, the rapidly-sedimenting magnetic particles can be collected at an isolation site. The isolation site is a location in the container that is proximate to the magnet. In some aspects, the isolation site is at or proximate to the bottom of the container. The rapidly-sedimenting magnetic particles described herein allow for rapid mixing and isolation. For example, mixing and collecting the rapidly-sedimenting magnetic particles at the isolation site can take less than 60 seconds, less than 30 seconds, in a range of from about 5 seconds to about 60 seconds, about 10 seconds to about 50 seconds, about 20 seconds to about 40 seconds, less than, equal to, or greater than about 5 seconds, 10, 15, 20, 25, 30, 35, 40, 45, 50, 55, or about 60 seconds. In some aspects isolating the rapidly-sedimenting magnetic particles alone can take less than 60 seconds, less than 30 seconds, in a range of from about 5 seconds to about 60 seconds, about 10 seconds to about 50 seconds, about 20 seconds to about 40 seconds, less than, equal to, or greater than about 5 seconds, 10, 15, 20, 25, 30, 35, 40, 45, 50, 55, or about 60 seconds. The isolation step is inclusive of the time it takes from the point at which the rapidly-sedimenting magnetic particles are exposed to the magnetic field of the magnet to the point where at least about 50 wt % of the rapidly-sedimenting magnetic particles in a container are located at the isolation site, at least about 60 wt % of the rapidly-sedimenting magnetic particles in a container are located at the isolation site, at least about 70 wt % of the rapidly-sedimenting magnetic particles in a container are located at the isolation site, at least about 80 wt % of the rapidly-sedimenting magnetic particles in a container are located at the isolation site, at least about 90 wt % of the rapidly-sedimenting magnetic particles in a container are located at the isolation site, or about 100 wt % of the rapidly-sedimenting magnetic particles in a container are located at the isolation site.

After the rapidly-sedimenting magnetic particles are collected at the isolation site, the resulting supernatant can be removed from the container. Removal can be facilitated, for example, using the pipettes. Alternatively, the magnetic particles can be separated and removed from the container using an insertable magnetic rod. The isolated rapidly-sedimenting magnetic particles can be contacted with an elution buffer to elute any analytes from the rapidly-sedimenting magnetic particle. For example, if the rapidly-sedimenting magnetic particles may have nucleic acids bound to the outer coating layer, water can be contacted with the rapidly-sedimenting magnetic particles to elute the nucleic acid.

In examples where the analyte of interest includes a nucleic acid, the eluted nucleic acid can be removed and subjected to a polymerase chain reaction (PCR) procedure to amplify any target nucleic acid present. If the target nucleic acid is RNA, the PCR procedure can be a reverse transcription PCR procedure. The system can be capable of mixing the eluted nucleic acid with any reagents or materials needed to conduct the PCR procedure. For example a buffer, primer, or enzyme can be pipetted into the container including the eluted nucleic acid. Moreover, in some aspects, the container can be moved to another location on the system that includes a thermocycler for conducting the PCR procedure.

In some further examples, the sample processing system can also include an analyzer. In some examples, the analyzer can be operatively coupled to the container. It will be appreciated by those skilled in the art that any suitable analyzer can be used to analyze the analyte or the sample. The analyzer can include any suitable instrument that is capable of analyzing a sample such as a biological sample. Examples of analyzers include mass spectrometers, immunoanalyzers, hematology analyzers, microbiology analyzers, and/or molecular biology analyzers. PCT Application No. PCT/US2018/033927 discloses an integrated sample processing system with multiple detection capability, and is hereby incorporated by reference herein in its entirety.

In some examples, the analyzer can be an immunoanalyzer used for detecting a label (chemoluminescent, electrochemiluminescent, fluorescent, radioactive isotope, DNA, etc.) or using a label free system. Other types of analyzers can include hematology analyzers, microbiology analyzers, chemistry analyzers, urine analyzers, biochemical analyzers, and/or a molecular biology analyzers. When analyzing a biological sample, one or more of these types of analyzers, in any suitable combination, can be used to analyze the biological sample.

A hematology analyzer can be used to perform complete blood counts, erythrocyte sedimentation rates (ESRs), and/or coagulation tests. Automated cell counters sample the blood, and quantify, classify, and describe cell populations using both electrical and optical techniques.

A microbiology analyzer can function as a diagnostic tool for determining the identity of a biological organism. In some examples, a microbiology analyzer can identify an infecting microorganism. Such analyzers can use biochemicals in a plurality of small sample test microwells in centrifugal rotors that contain different substrates, or in multi-well panels, depending on the type of test being performed.

A molecular biology analyzer can be a device which can analyze a biological sample at its molecular level. An example of a molecular biology analyzer can include a nucleic acid analyzer such as a DNA analyzer.

A chemistry analyzer can run assays on clinical samples such as blood serum, plasma, urine, and cerebrospinal fluid to detect the presence of analytes relating to disease or drugs. A chemistry analyzer can use photometry. In photometry, a sample is mixed with the appropriate reagent to produce a reaction that results in a color. The concentration of the analyte determines the strength of color produced. The photometer shines light of the appropriate wavelength at the sample and measures the amount of light absorbed, which is directly correlated to the concentration of the analyte in the sample. Another analytical method used in a chemistry analyzer is the use of ion selective electrodes (ISE) to measure ions such as Na⁺, K⁺, Cl⁻, and Li⁺. An ISE is a sensor that determines the concentration of ions in a solution by measuring the current flow through an ion selective membrane.

A “mass spectrometer” is an instrument which can measure the masses and relative concentrations of atoms and molecules. One example of a mass spectrometer makes use of the basic magnetic force on a moving charged particle. Basically, the instrument ionizes a sample and then deflects the ions through a magnetic field based on the mass-to-charge ratio of the ion. The mass spectrum can then be used to determine the elemental or isotopic signature of a sample, the masses of particles and of molecules, and to elucidate the chemical structures of molecules, such as peptides and other chemical compounds. Commercially available mass spectrometers can be categorized based on how they sector mass selection, including time-of-flight, quadrupole MS, ion traps (including 3D quadrupole, cylindrical ion traps, linear quadrapole ion traps, orbitraps), Fourier transform ion cyclotron resonance (FTMS), etc. Alternatively, they can be sectored based on ion source (laser desorption, matrix assisted laser desorption, thermal ionization, plasma, spark source, etc.) or detectors (electron multipliers (such as Faraday cups and ion-to-photon detectors), inductive detectors, etc.). In an example, the mass spectrometer can be a triple quadrupole mass spectrometer.

In accordance with various aspects of the present teachings, a kit can include ferrimagnetic particles and a container. The ferrimagnetic particles can be disposed in the container. The kit can further comprise reagents for desired analytic methods. The reagent can be any suitable reagent (e.g., precipitating reagents, wash buffers, elution buffers, and the like) that can be used while processing or analyzing a sample, for example, analyzing the sample for the presence of a particular analyte, such a biological molecule. In other examples, the kit can further comprise any portion of the sample processing system, as described herein.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “weight-average molecular weight” as used herein refers to M_{w}, which is equal to ΣMᵢ²nᵢ/ΣMᵢnᵢ, where nᵢ is the number of molecules of molecular weight Mᵢ. In various examples, the weight-average molecular weight can be determined using light scattering, small angle neutron scattering, X-ray scattering, and sedimentation velocity.

As used herein, the term “polymer” refers to a molecule having at least one repeating unit and can include copolymers.

The polymers described herein can terminate in any suitable way. In some embodiments, the polymers can terminate with an end group that is independently chosen from a suitable polymerization initiator, —H, —OH, a substituted or unsubstituted (C₁-C₂₀) hydrocarbyl (e.g., (C₁-C₁₀)alkyl or (C₆-C₂₀) aryl) interrupted with 0, 1, 2, or 3 groups independently selected from —O—, substituted or unsubstituted —NH—, and —S—, a poly (substituted or unsubstituted (C₁-C₂₀) hydrocarbyloxy), and a poly (substituted or unsubstituted (C₁-C₂₀) hydrocarbylamino).

### EXAMPLES

### Protocol 1: Covalent Coupling of Ligands to Magnetic Beads

Magnetic beads (as used in the Examples, “magnetic beads” refer to magnetic particles supplied as part of the product available under the trade designation EMnetik™, available from Beckman Coulter, Brea, CA and sold as of June 2021) were activated by carbodiimide, 1-ethyl-3-(3-dimethylaminopropyl) carbodiimide (EDAC) or combination EDAC and sulfo N-hydroxy succinimide and then reacted with the free amino group of an enzyme, protein, antibody, fluorescein isothiocyanate (“FITC”) polyethylene glycol (“PEG”), an amine, or a mixture thereof. The resulting mixture was continuously mixed in the reaction mixture for the optimization of protein coupling on the bead surface. In the case where an enzyme is used, the number of equivalents of the enzyme used is optimized to get the optimal activity. The amount of the bound protein can be determined by a BCA assay. Covalent adsorption provides the reduction of contamination of the sample due to leaching of biomolecules.

### Protocol 2: Adsorption of Ligands to Magnetic Beads

Magnetic beads described herein at Protocol 1 are treated with a free amino group of an enzyme, protein, antibody, or a mixture thereof. Enzymes, proteins, antibodies, FITC, PEG, an amine, or a mixture thereof are continuously mixed in the reaction mixture for the optimization of protein coupling on the bead surface. Concentrations of any salts, ionic species, detergents and the pH of the mixture are controlled to enhance adsorption. The number of equivalents of the substrate used is optimized to get the optimal activity. The surface is treated the amount of the bound protein can be determined by BCA assay.

### Protocol 3: Covalent Coupling of Streptavidin to Magnetic Beads

The magnetic beads of Protocol 1 were first activated by carbodiimide, EDAC or combination of EDAC and sulfo N-hydroxy succinimide and then reacted with the free amino group of the lyophilized streptavidin. Enzymes are continuously mixed in the reaction mixture for the optimization of protein coupling on the bead surface. The number of equivalents of the streptavidin used is optimized to get the optimal activity. The amount of the bound protein can be determined by BCA assay.

Protocol 4: Functionalization of Magnetic Beads with Amino Groups

The magnetic beads of Protocol 1 were first activated by carbodiimide, 1-ethyl-3-(3-dimethylaminopropyl) carbodiimide (EDAC) or combination of EDAC and sulfo N-hydroxy succinimide and then reacted with the excess of ethylene diamine or polyethylene glycol diamine. The reaction is continuously mixed in the reaction mixture for the optimization of protein coupling on the bead surface. Number of equivalents of the EDAC/sulfo-NHS and diamine used are optimized to get the optimal activity. The percentage replacement of carboxylic surface by amino surface is confirmed by IR spectroscopy.

### Protocol 5: Assay for Trypsin and Proteinase K

Protease activity was measured using fluorescein thiocarbamoyl-casein derivative (FTC-casein). The FTC-casein was incubated with a trypsin-functionalized of proteinase K-functionalized magnetic particle produced according to Protocol 1 or Protocol 3. Trichloroacetic acid (TCA) was added to the reaction mixture to precipitate any remaining FTC-casein. The supernatant was collected following centrifugation and the FTC-peptides were quantified by measuring the absorbance at 492 nm. The intensity of the color produced is directly proportional to the total protease activity in the sample.

Thirty samples were tested for their ability to possess trypsin activity. Twenty-six of the thirty samples were found to possess trypsin activity. Samples having trypsin activity had intensity values above a minimum threshold value.

Seventy samples were tested for their ability to possess Proteinase K activity. All samples were found to possess Proteinase K activity. Samples having Proteinase K activity had intensity values above a minimum threshold value.

### Protocol 6: Assay for Streptavidin

Streptavidin activity was measured using a magnetic particle functionalized with streptavidin produced according to the Protocol 1 or Protocol 3. The streptavidin-functionalized magnetic particles were mixed with biotinylated antibodies (anti-CD19) in 250 μl of phosphate-buffered saline (PBS) under stirring for one hour at 4° C. The resulting pellet was washed with 200 μl of PBS and this was repeated three times. The bound proteins were run on SDS-PAGE and stained for anti-CD19 protein. The SDS-PAGE showed that several magnetic particles were able to capture streptavidin.

### Protocol 7: Assay for Concentration of Virus

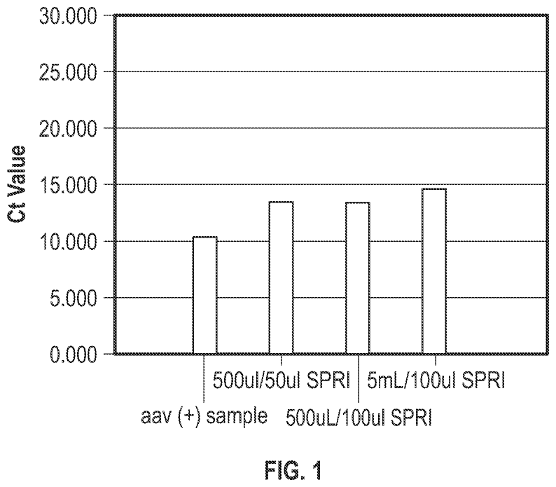

The ability of the magnetic particles to capture a virus was measured by mixing 50 μl of 2×10¹¹ particles/ml adeno-associated virus with 100 μl of the magnetic particles. The mixture was pelleted on a magnet and washed with 500 μl of PBS, and resuspended. This was repeated three times. The amount of virus captured was determined using qPCR. FIG. 1 is a graph that shows that the magnetic beads can concentrate virus from a larger volume of material.

### Exemplary Aspects

The following exemplary aspects are provided, the numbering of which is not to be construed as designating levels of importance:

Aspect 1 provides a method of processing a sample in a solution, the method comprising:
contacting the sample with a rapidly-sedimenting magnetic particle including a ferrimagnetic material and a ligand, wherein the ligand selectively interacts with a target molecule in the sample, and wherein:
a density of the rapidly-sedimenting magnetic particle is in a range of from about 1.1 g/cm³ to about 5.2 g/cm³; and
a remanence of the rapidly-sedimenting magnetic particle is in a range of from about 0 emu/g to about 20 emu/g.

Aspect 2 provides a method of processing a sample in a solution, the method comprising:
contacting the sample with a rapidly-sedimenting magnetic particle including a ferrimagnetic material and a ligand, wherein the ligand selectively interacts with a target molecule in the sample, and wherein:
a density of the rapidly-sedimenting magnetic particle is in a range of from about 1.1 g/cm³ to about 5.2 g/cm³; and
a size of the rapidly-sedimenting magnetic particle is in a range of from about 75 nm to about 200 nm.

Aspect 3 provides a method of processing a sample in a solution, the method comprising:
contacting the sample with a rapidly-sedimenting magnetic particle including a ferrimagnetic material and a ligand, wherein the ligand selectively interacts with a target molecule in the sample, and wherein:
a density of the rapidly-sedimenting magnetic particle is in a range of from about 1.1 g/cm³ to about 5.2 g/cm³; and
a sedimentation velocity of the rapidly-sedimenting magnetic particle is in a range of from about 0.3 mm/s to about 50 mm/s when subjected to a magnetic field having a strength in a range of from about 750 Oersted to about 14,000 Oersted.

Aspect 4 provides the method of any of Aspects 1 to 2, wherein a sedimentation velocity of the rapidly-sedimenting magnetic particle is in a range of from about 0.5 mm/s to about 20 mm/s when subjected to a magnetic field having a strength in a range of from about 750 Oersted to about 14,000 Oersted

Aspect 5 provides the method of any of Aspects 1 or 3, wherein a size of the rapidly-sedimenting magnetic particle is in a range of from about 75 nm to about 200 nm.

Aspect 6 provides the method of any of Aspects 2 or 3, wherein a remanence of the rapidly-sedimenting magnetic particle is in a range of from about 0 emu/g to about 10 emu/g.

Aspect 7 provides the method of any of Aspects 1-6, wherein a maximum field strength of the rapidly-sedimenting magnetic particle ranges from about 35 emu to about 100 emu/g.

Aspect 8 provides a method of isolating nucleic acid from a sample in solution, the method comprising:
contacting the sample with the rapidly-sedimenting magnetic particle of any of Aspects 1-7, wherein the ligand comprises a carboxylic acid group, an amine group, an azide group, an alkane group, an alkene group, an alkyne group, a maleimide group, an acrylate group, an aldehyde group, a sulfhydryl group, an N-hydroxysuccinimide (NHS) activated group, an epoxy group, a hydroxyl groups, a sulfide, biotin, a bis-sulfone, a catechol, a cyclopentadiene, a norbornene, or a mixture thereof for the selective binding to target nucleic acids in the presence of a crowding or precipitating reagent, that selectively interacts with nucleic acids in the solution in the presence of a precipitating reagent.

Aspect 9 provides a system for isolating nucleic acid from a sample in solution, the system comprising:
a reagent including the rapidly-sedimenting magnetic particle of any of Aspects 1-7, wherein the ligand comprises a carboxylic acid group, an amine group, an azide group, an alkane group, an alkene group, an alkyne group, a maleimide group, an acrylate group, an aldehyde group, a sulfhydryl group, an N-hydroxysuccinimide (NHS) activated group, an epoxy group, a hydroxyl groups, a sulfide, biotin, a bis-sulfone, a catechol, a cyclopentadiene, a norbornene, or a mixture thereof for the selective binding to target nucleic acids in the presence of a crowding or precipitating reagent, that selectively interacts with nucleic acids in the solution in the presence of a precipitating reagent; and a liquid handler configured and programmed to isolate the COVID-19 from the sample solution using the reagent.

Aspect 10 provides a rapidly-sedimenting magnetic particle comprising:
a ferrimagnetic material, wherein
   a size of the rapidly-sedimenting magnetic particle is in a range of from about 75 nm to about 200 μm;
   a remanence of the rapidly-sedimenting magnetic particle is in a range of from about 0 emu/g to about 20 emu/g; and
   a density of the rapidly-sedimenting magnetic particle is in a range of from about 1.1 g/cm³ to about 5.2 g/cm³.

Aspect 11 provides a rapidly-sedimenting magnetic particle comprising:
a ferrimagnetic material, wherein
a sedimentation velocity of the rapidly-sedimenting magnetic particle is in a range of from about 0.3 mm/s to about 50 mm/s when subjected to a magnetic field having a strength in a range of from about 750 Oersted to about 14,000 Oersted; and
a remanence of the rapidly-sedimenting magnetic particle is in a range of from about 0 emu/g to about 20 emu/g.

Aspect 12 provides a rapidly-sedimenting magnetic particle comprising:
a density in a range of from about 1.1 g/cm³ to about 5.2 g/cm³; and
a sedimentation velocity of the rapidly-sedimenting magnetic particle is in a range of from about 0.3 mm/s to about 50 mm/s when subjected to a magnetic field having a strength in a range of from about 750 Oersted to about 14,000 Oersted; and
a remanence of the rapidly-sedimenting magnetic particle is in a range of from about 0 emu/g to about 20 emu/g.

Aspect 13 provides the rapidly-sedimenting magnetic particle of any one of Aspects 10-12, wherein a maximum field strength of the rapidly-sedimenting magnetic particle ranges from about 35 emu/g to about 100 emu/g.

Aspect 14 provides the rapidly-sedimenting magnetic particle of any one of Aspects 10-13, wherein the remanence of the rapidly-sedimenting magnetic particle ranges from about 0 emu/g to about 5 emu/g.

Aspect 15 provides the rapidly-sedimenting magnetic particle of any one of Aspects 10-14, wherein the ferrimagnetic material comprises Fe₃O₄.

Aspect 16 provides the rapidly-sedimenting magnetic particle of any one of Aspects 10-15, wherein a major dimension of the rapidly-sedimenting magnetic particle is from about 80 nm to about 150 nm.

Aspect 17 provides the rapidly-sedimenting magnetic particle of any one of Aspects 10-16, wherein a major dimension of the rapidly-sedimenting magnetic particle is from 95 nm to about 100 nm.

Aspect 18 provides the rapidly-sedimenting magnetic particle of any one of Aspects 16 or 17, wherein the size of the rapidly sedimenting magnetic particle is a major dimension of the rapidly sedimenting magnetic particle.

Aspect 19 provides the rapidly-sedimenting magnetic particle of any one of Aspects 10-18, wherein the core coating layer comprises SiO₂, TiO₂, ZnO₂, Al₂O₃, CeO₂, a ceramic, polyacrylic acid, poly(methyl acrylate), polystyrene, divinylbenzene, polyvinylpyrrolidone, polyvinyl alcohol, or a mixture thereof.

Aspect 20 provides the rapidly-sedimenting magnetic particle of any one of Aspects 10-19, wherein the core coating layer comprises SiO₂.

Aspect 21 provides the rapidly-sedimenting magnetic particle of any one of Aspects 10-20 further comprising a silane linker disposed between the core coating layer and the outer layer.

Aspect 22 provides the rapidly-sedimenting magnetic particle of Aspect 21, wherein the silane linker comprises N-(3-triethoxysilylpropyl) gluconamide).

Aspect 23 provides the rapidly-sedimenting magnetic particle of any one of Aspects 1-22, wherein the core comprises a surface area and the coating layer coats the entire surface area of the core.

Aspect 24 provides the rapidly-sedimenting magnetic particle of any one of Aspects 1-23, wherein at least a portion of the outer layer is functionalized.

Aspect 25 provides the rapidly-sedimenting magnetic particle of any one of Aspects 1-24, wherein the outer layer is functionalized with thiol, streptavidin, an amine, a hydroxyl, a tosyl, an epoxy, an alkyl, a vinyl, an aryl, an enzyme, a protein, a deoxyribonucleic acid, a ribonucleic acid, an immunoglobulin G, an immunoglobulin A, a carboxyl group, a monoclonal antibody, or combinations thereof.

Aspect 26 provides the rapidly-sedimenting magnetic particle of any one of Aspects 1-25 wherein the outer layer comprises a functionalized poly(methyl vinyl ether-alt-maleic anhydride).

Aspect 27 provides the rapidly-sedimenting magnetic particle of 26, wherein the poly(methyl vinyl ether-alt-maleic anhydride) has a weight-average molecular weight in a range of from about 200 kDa to about 500 kDa.

Aspect 28 provides the rapidly-sedimenting magnetic particle of any one of Aspects 1-27, wherein the outer layer comprises a carboxyl group, an enzyme, or a combination thereof.

Aspect 29 provides the rapidly-sedimenting magnetic particle of any one of Aspects 1-28, wherein the outer layer comprises a carboxyl.

Aspect 30 provides the rapidly-sedimenting magnetic particle of any one of Aspects 1-29, wherein the outer layer comprises an enzyme.

Aspect 31 provides the rapidly-sedimenting magnetic particle of Aspect 30, wherein the enzyme comprises a nucleic acid fragmentation enzyme.

Aspect 32 provides the rapidly-sedimenting magnetic particle of Aspect 31, wherein the nucleic acid fragmentation enzyme comprises a caspase-activated DNase, a fragmentase, a micrococcal nuclease, or a mixture thereof.

Aspect 33 provides the rapidly-sedimenting magnetic particle of any one of Aspects 31 or 32, wherein the enzyme comprises a lysing enzyme.

Aspect 35 provides the rapidly-sedimenting magnetic particle of Aspect 33, wherein the lysing enzyme comprises a lysozyme, a proteinase K, a collagenase, or a mixture thereof.

Aspect 36 provides the rapidly-sedimenting magnetic particle of any one of Aspects 1-35, wherein the outer layer comprises a combination of functional groups selected from an enzyme and a carboxyl group.

Aspect 37 provides a kit comprising the rapidly-sedimenting magnetic particle of any one of Aspects 1-36.

Aspect 38 provides the kit of Aspect 37, wherein the rapidly-sedimenting magnetic particle is a first rapidly-sedimenting magnetic particle and the kit further comprises a second rapidly-sedimenting magnetic particle, the first and second rapidly-sedimenting magnetic particles comprising different outer coating layers.

Aspect 39 provides the kit of Aspect 38, wherein the outer coating layer of the first rapidly-sedimenting magnetic particle comprises a plurality of carboxyl groups and the outer coating layer of the second rapidly-sedimenting magnetic particle comprises a plurality of enzymes.

Aspect 40 provides the kit of any one of Aspects 37-39, wherein the kit further comprises an analyte of interest.

Aspect 41 provides the kit of Aspect 40, wherein the analyte of interest comprises a nucleic acid, an enzyme, or a mixture thereof.

Aspect 42 provides the kit of Aspect 41, wherein the nucleic acid is a viral ribonucleic acid.

Aspect 43 provides the kit of Aspect 42, wherein the vial ribonucleic acid is from a coronavirus.

Aspect 44 provides the kit of 43, wherein the nucleic acid is a ribonucleic acid of a coronavirus that causes severe acute respiratory syndrome.

Aspect 46 provides a method of processing a sample, the method comprising:
contacting the sample with the rapidly-sedimenting magnetic particle of any one of Aspects 1-44.

Aspect 47 provides the method of Aspect 46, wherein the solution comprises an analyte of interest.

Aspect 48 provides the method of any one of Aspects 46 or 47, further comprising subjecting the rapidly-sedimenting magnetic particle to a magnetic field, thereby isolating the rapidly-sedimenting magnetic particle.

Aspect 49 provides the method of Aspect 48, wherein subjecting the rapidly-sedimenting magnetic particle to a magnetic field comprises reversibly positioning a magnet proximate to the sample.

Aspect 50 provides the method of Aspect 49, wherein the magnet is reversibly activated using a controller.

Aspect 51 provides the method of any one of Aspects 48 or 50, wherein the magnet is a permanent magnet.

Aspect 52 provides the method of any one of Aspects 48-51, wherein the magnet is reversibly positioned underneath the sample.

Aspect 53 provides the method of any one of Aspects 48-52, wherein the analyte of interest is a nucleic acid.

Aspect 54 provides the method of Aspect 53, wherein the nucleic acid is viral ribonucleic acid.

Aspect 55 provides the method of Aspect 54, wherein the vial ribonucleic acid is from a coronavirus.

Aspect 56 provides the method of 55, wherein the nucleic acid is a ribonucleic acid of a virus causing severe acute respiratory syndrome.

Aspect 57 provides the method of any one of Aspects 48-56, wherein the analyte of interest is a substrate of the enzyme.

Aspect 58 provides the method of any one of Aspects 48-57, wherein contacting the rapidly-sedimenting magnetic particle and the solution comprising the analyte of interest comprises mixing the rapidly-sedimenting magnetic particle and the solution.

Aspect 59 provides the method of Aspect 58, wherein mixing is conducted by aspiration.

Aspect 60 provides the method of Aspect 59, wherein mixing comprises exposing the rapidly-sedimenting magnetic particle to a magnetic field and isolating the rapidly-sedimenting magnetic particle after the rapidly-sedimenting magnetic particle is exposed to a magnetic field.

Aspect 61 provides the method of Aspect 60, wherein mixing takes less than 60 seconds.

Aspect 62 provides the method of any one of Aspects 60 or 61, wherein mixing takes less than 30 seconds.

Aspect 63 provides the method of any one of Aspects 60-62, wherein mixing takes 5 seconds to 60 seconds.

Aspect 64 provides the method of any one of Aspects 60-63, wherein the rapidly-sedimenting magnetic particles are moved vertically to an isolation site.

Aspect 65 provides the method of any one of Aspects 60-64, further comprising removing the rapidly-sedimenting magnetic particles from the solution.

Aspect 66 provides the method of Aspect 65, further comprising eluting the analyte of interest from the rapidly-sedimenting magnetic particle.

Aspect 67 provides the method of Aspect 66, further comprising performing a polymerase chain reaction procedure on the analyte of interest.

Aspect 68 provides the method of Aspect 67, wherein the polymerase chain reaction procedure is a reverse transcription polymerase chain reaction procedure.

Aspect 69 provides the method of any one of Aspects 48-68, wherein the method is a method of detecting a virus causing severe acute respiratory syndrome.

Aspect 70 provides the method of any one of Aspects 48-69, wherein the method or a portion thereof is conducted using an automated pipetting machine.

Aspect 71 provides a method of processing a sample, the method comprising: providing the rapidly-sedimenting magnetic particle of any one of Aspects 10-70; contacting a solution containing an analyte of interest with the rapidly-sedimenting magnetic particle;
subjecting the rapidly-sedimenting magnetic particle to a magnetic field, thereby allowing the rapidly-sedimenting magnetic particle to be separated from the solution
collecting the rapidly-sedimenting magnetic particle at a collection site after the rapidly-sedimenting magnetic particle is exposed to a magnetic field;
isolating the rapidly-sedimenting magnetic particles;
eluting the analyte of interest from the rapidly-sedimenting magnetic particle; and performing a polymerase chain reaction procedure on the analyte of interest.

Aspect 72 provides the method of any one of Aspects 46-71, wherein the ligand is a reactant for a chemical reaction with the analyte of interest.

Aspect 73 provides the method of Aspect 72, wherein the chemical reaction comprises cleaving the analyte of interest or combining the analyte of interest with another compound.

Aspect 74 provides the method of any one of Aspects 46-73, wherein the analyte of interest is a drug or prodrug.

Aspect 75 provides the method of any one of Aspects 46-74, wherein the analyte of interest is fluorescent active or UV active.

Aspect 76 provides the method of any one of Aspects 46-75, wherein the method of processing comprises using the magnetic particle as a starting material or a catalyst in a synthetic reaction.

Aspect 77 provides the method of Aspect 76, wherein the magnetic particle is not present in final product formed from the synthetic reaction.

Aspect 78 provides the method of any one of Aspects 46-77, wherein the analyte of interest is a virus.

Aspect 79 provides the magnetic particle of any one of Aspects 1-78, wherein the magnetic particle is adapted to remain suspended in a solution and be free of magnetically induced aggregation.

What is claimed is:

1. A method of processing a sample in a solution, the method comprising:
contacting the sample with a rapidly-sedimenting magnetic particle including a ferrimagnetic material and a ligand, wherein the ligand selectively interacts with a target molecule in the sample, and wherein:
a density of the rapidly-sedimenting magnetic particle is in a range of from about 1.1 g/cm³ to about 5.2 g/cm³; and
a remanence of the rapidly-sedimenting magnetic particle is in a range of from about 0 emu/g to about 20 emu/g.

2. A method of processing a sample in a solution, the method comprising:
contacting the sample with a rapidly-sedimenting magnetic particle including a ferrimagnetic material and a ligand, wherein the ligand selectively interacts with a target molecule in the sample, and wherein:
a density of the rapidly-sedimenting magnetic particle is in a range of from about 1.1 g/cm³ to about 5.2 g/cm³; and
a size of the rapidly-sedimenting magnetic particle is in a range of from about 75 nm to about 200 nm.

3. A method of processing a sample in a solution, the method comprising:
contacting the sample with a rapidly-sedimenting magnetic particle including a ferrimagnetic material and a ligand, wherein the ligand selectively interacts with a target molecule in the sample, and wherein:
a density of the rapidly-sedimenting magnetic particle is in a range of from about 1.1 g/cm³ to about 5.2 g/cm³; and
a sedimentation velocity of the rapidly-sedimenting magnetic particle is in a range of from about 0.3 mm/s to about 50 mm/s when subjected to a magnetic field having a strength in a range of from about 750 Oersted to about 14,000 Oersted.

4. The method of any of claims 1 to 2, wherein a sedimentation velocity of the rapidly-sedimenting magnetic particle is in a range of from about 0.5 mm/s to about 20 mm/s when subjected to a magnetic field having a strength in a range of from about 750 Oersted to about 14,000 Oersted

5. The method of any of claim 1 or 3, wherein a size of the rapidly-sedimenting magnetic particle is in a range of from about 75 nm to about 200 nm.

6. The method of any of claim 2 or 3, wherein a remanence of the rapidly-sedimenting magnetic particle is in a range of from about 0 emu/g to about 10 emu/g.

7. The method of any of claims 1-6, wherein a maximum field strength of the rapidly-sedimenting magnetic particle ranges from about 35 emu to about 100 emu/g.

8. A method of isolating nucleic acid from a sample in solution, the method comprising:
contacting the sample with the rapidly-sedimenting magnetic particle of any of claims 1-7, wherein the ligand comprises a carboxylic acid group, an amine group, an azide group, an alkane group, an alkene group, an alkyne group, a maleimide group, an acrylate group, an aldehyde group, a sulfhydryl group, an N-hydroxysuccinimide (NHS) activated group, an epoxy group, a hydroxyl groups, a sulfide, biotin, a bis-sulfone, a catechol, a cyclopentadiene, a norbornene, or a mixture thereof for the selective binding to target nucleic acids in the presence of a crowding or precipitating reagent that selectively interacts with nucleic acids in the solution in the presence of a precipitating reagent.

9. A system for isolating nucleic acid from a sample in solution, the system comprising:
a reagent including the rapidly-sedimenting magnetic particle of any of claims 1-7, wherein the ligand comprises a carboxylic acid group, an amine group, an azide group, an alkane group, an alkene group, an alkyne group, a maleimide group, an acrylate group, an aldehyde group, a sulfhydryl group, an N-hydroxysuccinimide (NHS) activated group, an epoxy group, a hydroxyl groups, a sulfide, biotin, a bis-sulfone, a catechol, a cyclopentadiene, a norbornene, or a mixture thereof for the selective binding to target nucleic acids in the presence of a crowding or precipitating reagent that selectively interacts with nucleic acids in the solution in the presence of a precipitating reagent; and
a liquid handler configured and programmed to isolate the COVID-19 from the sample solution using the reagent.

10. A rapidly-sedimenting magnetic particle comprising:
a ferrimagnetic material, wherein
a size of the rapidly-sedimenting magnetic particle is in a range of from about 75 nm to about 200 μm;
a remanence of the rapidly-sedimenting magnetic particle is in a range of from about 0 emu/g to about 20 emu/g; and
a density of the rapidly-sedimenting magnetic particle is in a range of from about 1.1 g/cm³ to about 5.2 g/cm³.

11. A rapidly-sedimenting magnetic particle comprising:
a ferrimagnetic material, wherein
a sedimentation velocity of the rapidly-sedimenting magnetic particle is in a range of from about 0.3 mm/s to about 50 mm/s when subjected to a magnetic field having a strength in a range of from about 750 Oersted to about 14,000 Oersted; and
a remanence of the rapidly-sedimenting magnetic particle is in a range of from about 0 emu/g to about 20 emu/g.

12. A rapidly-sedimenting magnetic particle comprising:
a density in a range of from about 1.1 g/cm³ to about 5.2 g/cm³; and
a sedimentation velocity of the rapidly-sedimenting magnetic particle is in a range of from about 0.3 mm/s to about 50 mm/s when subjected to a magnetic field having a strength in a range of from about 750 Oersted to about 14,000 Oersted; and
a remanence of the rapidly-sedimenting magnetic particle is in a range of from about 0 emu/g to about 20 emu/g.

13. The rapidly-sedimenting magnetic particle of any one of claims 10-12, wherein a maximum field strength of the rapidly-sedimenting magnetic particle ranges from about 35 emu/g to about 100 emu/g.

14. The rapidly-sedimenting magnetic particle of any one of claims 10-13, wherein the remanence of the rapidly-sedimenting magnetic particle ranges from about 0 emu/g to about 5 emu/g.

15. The rapidly-sedimenting magnetic particle of any one of claims 10-14, wherein the ferrimagnetic material comprises Fe₃O₄.

16. The rapidly-sedimenting magnetic particle of any one of claims 10-15, wherein a major dimension of the rapidly-sedimenting magnetic particle is from about 80 nm to about 150 nm.

17. The rapidly-sedimenting magnetic particle of any one of claims 10-16, wherein a major dimension of the rapidly-sedimenting magnetic particle is from 95 nm to about 100 nm.

18. The rapidly-sedimenting magnetic particle of any one of claim 16 or 17, wherein the size of the rapidly sedimenting magnetic particle is a major dimension of the rapidly sedimenting magnetic particle.

19. The rapidly-sedimenting magnetic particle of any one of claims 10-18, wherein the core coating layer comprises SiO₂, TiO₂, ZnO₂, Al₂O₃, CeO₂, a ceramic, polyacrylic acid, poly(methyl acrylate), polystyrene, divinylbenzene, polyvinylpyrrolidone, polyvinyl alcohol, or a mixture thereof.

20. The rapidly-sedimenting magnetic particle of any one of claims 10-19, wherein the core coating layer comprises SiO₂.

21. The rapidly-sedimenting magnetic particle of any one of claims 10-20 further comprising a silane linker disposed between the core coating layer and the outer layer.

22. The rapidly-sedimenting magnetic particle of claim 21, wherein the silane linker comprises N-(3-triethoxysilylpropyl) gluconamide).

23. The rapidly-sedimenting magnetic particle of any one of claims 10-22, wherein the core comprises a surface area and the coating layer coats the entire surface area of the core.

24. The rapidly-sedimenting magnetic particle of any one of claims 10-23, wherein at least a portion of the outer layer is functionalized.

25. The rapidly-sedimenting magnetic particle of any one of claims 10-24, wherein the outer layer is functionalized with thiol, streptavidin, an amine, a hydroxyl, a tosyl, an epoxy, an alkyl, a vinyl, an aryl, an enzyme, a protein, a deoxyribonucleic acid, a ribonucleic acid, an immunoglobulin G, an immunoglobulin A, a carboxyl group, a monoclonal antibody, or combinations thereof.

26. The rapidly-sedimenting magnetic particle of any one of claims 10-25 wherein the outer layer comprises a functionalized poly(methyl vinyl ether-alt-maleic anhydride).

27. The rapidly-sedimenting magnetic particle of 26, wherein the poly(methyl vinyl ether-alt-maleic anhydride) has a weight-average molecular weight in a range of from about 200 kDa to about 500 kDa.

28. The rapidly-sedimenting magnetic particle of any one of claims 10-27, wherein the outer layer comprises a carboxyl group, an enzyme, or a combination thereof.

29. The rapidly-sedimenting magnetic particle of any one of claims 10-28, wherein the outer layer comprises a carboxyl.

30. The rapidly-sedimenting magnetic particle of any one of claims 10-29, wherein the outer layer comprises an enzyme.

31. The rapidly-sedimenting magnetic particle of claim 30, wherein the enzyme comprises a nucleic acid fragmentation enzyme.

32. The rapidly-sedimenting magnetic particle of claim 31, wherein the nucleic acid fragmentation enzyme comprises a caspase-activated DNase, a fragmentase, a micrococcal nuclease, or a mixture thereof.

33. The rapidly-sedimenting magnetic particle of any one of claim 31 or 32, wherein the enzyme comprises a lysing enzyme.

34. The rapidly-sedimenting magnetic particle of claim 33, wherein the lysing enzyme comprises a lysozyme, a proteinase K, a collagenase, or a mixture thereof.

35. The rapidly-sedimenting magnetic particle of claim 34, wherein the lysing enzyme comprises a proteinase K.

36. The rapidly-sedimenting magnetic particle of any one of claims 10-35, wherein the outer layer comprises a combination of functional groups selected from an enzyme and a carboxyl group.

37. A kit comprising the rapidly-sedimenting magnetic particle of any one of 10-36.

38. The kit of claim 37, wherein the rapidly-sedimenting magnetic particle is a first rapidly-sedimenting magnetic particle and the kit further comprises a second rapidly-sedimenting magnetic particle, the first and second rapidly-sedimenting magnetic particles comprising different outer coating layers.

39. The kit of claim 38, wherein the outer coating layer of the first rapidly-sedimenting magnetic particle comprises a plurality of carboxyl groups and the outer coating layer of the second rapidly-sedimenting magnetic particle comprises a plurality of enzymes.

40. The kit of any one of claims 37-39, wherein the kit further comprises an analyte of interest.

41. The kit of claim 40, wherein the analyte of interest comprises a nucleic acid, an enzyme, or a mixture thereof.

42. The kit of claim 41, wherein the nucleic acid is a viral ribonucleic acid.

43. The kit of claim 42, wherein the vial ribonucleic acid is from a coronavirus.

44. The kit of 43, wherein the nucleic acid is a ribonucleic acid of a coronavirus that causes severe acute respiratory syndrome.

45. The kit of 44, wherein the nucleic acid is a ribonucleic acid of a coronavirus that causes COVID-19.

46. A method of processing a sample, the method comprising:
contacting the sample with the rapidly-sedimenting magnetic particle of any one of claims 10-44.

47. The method of claim 46, wherein the solution comprises an analyte of interest.

48. The method of any one of claim 46 or 47, further comprising subjecting the rapidly-sedimenting magnetic particle to a magnetic field, thereby isolating the rapidly-sedimenting magnetic particle.

49. The method of claim 48, wherein subjecting the rapidly-sedimenting magnetic particle to a magnetic field comprises reversibly positioning a magnet proximate to the sample.

50. The method of claim 49, wherein the magnet is reversibly activated using a controller.

51. The method of any one of claim 48 or 50, wherein the magnet is a permanent magnet.

52. The method of any one of claims 48-51, wherein the magnet is reversibly positioned underneath the sample.

53. The method of any one of claims 48-52, wherein the analyte of interest is a nucleic acid.

54. The method of claim 53, wherein the nucleic acid is viral ribonucleic acid.

55. The method of claim 54, wherein the vial ribonucleic acid is from a coronavirus.

56. The method of 55, wherein the nucleic acid is a ribonucleic acid of a virus causing severe acute respiratory syndrome.

57. The method of any one of claims 48-56, wherein the analyte of interest is a substrate of the enzyme.

58. The method of any one of claims 48-57, wherein contacting the rapidly-sedimenting magnetic particle and the solution comprising the analyte of interest comprises mixing the rapidly-sedimenting magnetic particle and the solution.

59. The method of claim 58, wherein mixing is conducted by aspiration.

60. The method of claim 59, wherein mixing comprises exposing the rapidly-sedimenting magnetic particle to a magnetic field and isolating the rapidly-sedimenting magnetic particle after the rapidly-sedimenting magnetic particle is exposed to a magnetic field.

61. The method of claim 60, wherein mixing takes less than 60 seconds.

62. The method of any one of claim 60 or 61, wherein mixing takes less than 30 seconds.

63. The method of any one of claims 60-62, wherein mixing takes 5 seconds to 60 seconds.

64. The method of any one of claims 60-63, wherein the rapidly-sedimenting magnetic particles are moved vertically to an isolation site.

65. The method of any one of claims 60-64, further comprising removing the rapidly-sedimenting magnetic particles from the solution.

66. The method of claim 65, further comprising eluting the analyte of interest from the rapidly-sedimenting magnetic particle.

67. The method of claim 66, further comprising performing a polymerase chain reaction procedure on the analyte of interest.

68. The method of claim 67, wherein the polymerase chain reaction procedure is a reverse transcription polymerase chain reaction procedure.

69. The method of any one of claims 48-68, wherein the method is a method of detecting a virus causing severe acute respiratory syndrome.

70. The method of any one of claims 48-69, wherein the method or a portion thereof is conducted using an automated pipetting machine.

71. A method of processing a sample, the method comprising:
providing the rapidly-sedimenting magnetic particle of any one of claims 10-70;
contacting a solution containing an analyte of interest with the rapidly-sedimenting magnetic particle;
subjecting the rapidly-sedimenting magnetic particle to a magnetic field, thereby allowing the rapidly-sedimenting magnetic particle to be separated from the solution collecting the rapidly-sedimenting magnetic particle at a collection site after the rapidly-sedimenting magnetic particle is exposed to a magnetic field;
isolating the rapidly-sedimenting magnetic particles;
eluting the analyte of interest from the rapidly-sedimenting magnetic particle; and
performing a polymerase chain reaction procedure on the analyte of interest.

72. The method of any one of claims 46-71, wherein the ligand is a reactant for a chemical reaction with the analyte of interest.

73. The method of claim 72, wherein the chemical reaction comprises cleaving the analyte of interest or combining the analyte of interest with another compound.

74. The method of any one of claims 46-73, wherein the analyte of interest is a drug or prodrug.

75. The method of any one of claims 46-74, wherein the analyte of interest is fluorescent active or UV active.

76. The method of any one of claims 46-75, wherein the method of processing comprises using the rapidly-sedimenting magnetic particle as a starting material or a catalyst in a synthetic reaction.

77. The method of claim 76, wherein the rapidly-sedimenting magnetic particle is not present in final product formed from the synthetic reaction.

78. The method of any one of claims 46-77, wherein the analyte of interest is a virus.

79. The magnetic particle of any one of claims 1-78, wherein the rapidly-sedimenting magnetic particle is adapted to remain suspended in a solution and be free of magnetically induced aggregation.
